(19) United States
(12) Patent Application Publication
(10) Pub. No.: US 2024/0425939 A1
(43) Pub. Date: Dec. 26, 2024

(54) TRANSGENIC MAIZE EVENT LP026-2 AND DETECTION METHOD THEREFOR

(71) Applicant: LONGPING BIOTECH (HAINAN) CO., LTD., Sanya, Hainan (CN)

(72) Inventors: Zhiwei JIA, Sanya (CN); Yuping LU, Sanya (CN); Xiaojiao LI, Sanya (CN); Qiang WANG, Sanya (CN); Shuxiu LI, Sanya (CN); Yu SUN, Sanya (CN); Jianhai WANG, Sanya (CN); Liyuan ZHAO, Sanya (CN); Xiaoyan SHI, Sanya (CN); Tao LI, Sanya (CN)

(73) Assignee: LONGPING BIOTECH (HAINAN) CO., LTD., Sanya, Hainan (CN)

(21) Appl. No.: 18/699,269

(22) PCT Filed: Jun. 25, 2023
(86) PCT No.: PCT/CN2023/102064

§ 371 Date: Apr. 6, 2024

(30) Foreign Application Priority Data
Sep. 23, 2022 (CN) 202211161744.2

(51) Int. Cl. C12Q 1/6895; C07K 14/325; C12N 9/10; C12N 15/82

(52) U.S. Cl. CPC C12Q 1/6895; C07K 14/325; C12N 9/1092; C12N 15/8275; C12N 15/8286; C12Q 2600/13

(57) ABSTRACT

The present invention provides a nucleic acid sequence, which comprises one or more selected from the sequences consisting of SEQ ID NOs: 1-7 and the complementary sequences thereof, the nucleic acid sequence is derived from plants, seeds or cells comprising the transgenic maize event LP026-2, and the representative sample of the seeds comprising the event having been deposited under deposit number CCTCC NO: P202207.

### CROSS-REFERENCE TO RELATED APPLICATION

This patent application is a national stage application of International Patent Application No. PCT/CN2023/102064, filed on Jun. 25, 2023, which claims priority to Chinese Patent Application No. 202211161744.2 entitled “Transgenic maize event LP026-2 and detection method therefor” filed on Sep. 23, 2022, the entire disclosure of which is incorporated to the present application by reference in its entirety.

### REFERENCE TO SEQUENCE LISTING

A computer readable xml file entitled “Sequence Listing”, that was created on Mar. 14, 2024, with a file size of 65,642 bytes, contains the sequence listing for this application, has been filed with this application, and is hereby incorporated by reference in its entirety.

### TECHNICAL FIELD

The present invention belongs to the technical field of molecular biology, and relates to a detection method of transgenic plants and processed products thereof, in particular to a transgenic maize event LP026-2 which is resistant to insects and tolerant to glyphosate herbicide application, and a nucleic acid sequence and a method for detecting the transgenic maize event LP026-2.

### BACKGROUND

Maize (Zea mays L.) is a main food crop in many parts of the world. Biotechnology has been applied to maize to improve its agronomic characters and quality. Insect resistance is one of the important agronomic characters in maize production, especially the resistance to Lepidoptera insects (such as Ostrinia furnacalis, Helicoverpa armigera, Spodoptera frugiperda, Mythimna seperataseparata and the like). The resistance of maize to Lepidoptera insects may be obtained by expressing an insect-resistant gene for Lepidoptera in maize plants using a transgenic method. Another important agronomic character is herbicide tolerance, especially glyphosate herbicide tolerance. The tolerance of maize to glyphosate herbicide may be obtained by expressing a glyphosate herbicide-tolerant gene (e.g., epsps) in maize plants by a transgenic method.

The expression of exogenous genes in plants is known to be influenced by the position where they are inserted into the maize genome, which may be due to the chromatin structure (e.g. heterochromatin) or proximity of transcriptional regulatory elements (e.g. enhancers) to integration sites. For this reason, it is often necessary to screen a large number of events before it is possible to identify commercially available events (i.e., events in which the introduced target gene is optimally expressed). For example, it has been observed that the expression levels of introduced genes in plants and other organisms may vary greatly between events; there may also be differences in the spatial or temporal patterns of expression, such as differences in the relative expression of transgenes between different plant tissues, which is manifested in the fact that the actual expression pattern may be inconsistent with the expected expression pattern of transcription regulatory elements in the introduced gene construct. Therefore, it is often necessary to generate hundreds of different events and screen out a single event with the expected expression level and pattern of the transgene for commercialization purposes. Such a transformation event has excellent resistance to Lepidoptera pests (e.g., Ostrinia furnacalis, Spodoptera frugiperda, Mythimna separata, Helicoverpa armigera, Agrotis ipsilon, Conogethes punctiferalis and the likes) and to glyphosate herbicide without compromising maize yields, and the transgenic traits can be backcrossed into other genetic backgrounds through hybridization using conventional breeding methods. The progeny produced by such hybridization method maintain the expression characteristics of the transgene and trait performance of the original transformants. Application of this strategic model ensures reliable gene expression in many varieties with stable resistance to Lepidoptera pests (e.g., Ostrinia furnacalis, Spodoptera frugiperda, Mythimna separata, Helicoverpa armigera, Agrotis ipsilon, Conogethes punctiferalis and the likes) and to glyphosate herbicide, which provides protection from major Lepidopteran pests and broad-spectrum weed control, while being well adapted to local growing conditions.

It will be beneficial to be able to detect the specific events to determine whether the progeny of sexual hybridization contain the target gene. In addition, the method of detecting specific events will also assist in complying with regulations, for example, the need for food derived from recombinant crops to be formally approved and labeled before being placed on the market. It is possible to detect the transgenes by any well-known polynucleotide detection assays, such as polymerase chain reaction (PCR) or DNA hybridization using polynucleotide probes. These detection methods usually focus on common genetic elements, such as promoters, terminators, marker genes and so on. Therefore, unless the sequence of chromosomal DNA adjacent to the inserted transgenic DNA (“flanking DNA”) is known, the above method cannot be used to distinguish different events, especially those generated with the same DNA construct. Therefore, it is now common to identify specific transgenic events by PCR using a pair of primers spanning the junction site between the inserted T-DNA and the flanking DNA, specifically a first primer containing the flanking sequence and a second primer containing the insertion sequence.

### SUMMARY

It is an object of the present invention to provide a transgenic maize event LP026-2 as well as a nucleic acid sequence for detecting the maize plant LP026-2 event and a detection method therefor, which is capable of identifying whether a biological sample contains a DNA molecule of the specific transgenic maize event LP026-2 quickly and accurately.

To achieve the above objectives, a nucleic acid sequence, which contains one or more selected from the sequences consisting of SEQ ID NOs: 1-7 (i.e. SEQ ID NO:1, SEQ ID NO:2, SEQ ID NO:3, SEQ ID NO:4, SEQ ID NO:5, SEQ ID NO:6, SEQ ID NO:7) and the complementary sequences thereof, is provided by the present invention. In some embodiments, the nucleic acid sequence is derived from plants, seeds or cells containing the maize event LP026-2, and the representative sample of the seeds containing the event was deposited at the China Center for Type Culture Collection (CCTCC for short, Address: Wuhan University Collection Center, No. 299 Bayi Road, Wuchang District, Wuhan City, Hubci Province, Postcode: 430072) on Apr. 15, 2022. In some embodiments, the nucleic acid sequence is an amplicon for diagnosing the maize event LP026-2.

In some embodiments of the present invention, the present invention provides a nucleic acid sequence comprising at least 11 continuous nucleotides in SEQ ID NO:3 or a complementary sequence thereof, and/or at least 11 continuous nucleotides in SEQ ID NO:4 or a complementary sequence thereof. In some embodiments, the nucleic acid sequence comprises SEQ ID NO:1 or a complementary sequence thereof, and/or SEQ ID NO:2 or a complementary sequence thereof. In some embodiments, the nucleic acid sequence comprises SEQ ID NO:3 or a complementary sequence thereof, and/or SEQ ID NO:4 or a complementary sequence thereof. In some embodiments, the nucleic acid sequence comprises SEQ ID NO:5 or a complementary sequence thereof.

The SEQ ID NO: 1 or a complementary sequence thereof is a sequence with a length of 22 nucleotides located near the insertion junction site at the 5′ end of the insertion sequence in the transgenic maize event LP026-2, which spans the genomic DNA sequence flanking the maize insertion site and the DNA sequence at the 5′ end of the insertion sequence, and the inclusion of the SEQ ID NO: 1 or a complementary sequence thereof can be identified as the presence of the transgenic maize event LP026-2. The SEQ ID NO:2 or a complementary sequence thereof is a sequence with a length of 22 nucleotides located near the insertion junction site at the 3′ end of the insertion sequence in the transgenic maize event LP026-2, which spans the DNA sequence at the 3′ end of the insertion sequence and the genomic DNA sequence flanking the maize insertion site, and the inclusion of the SEQ ID NO:2 or a complementary sequence thereof can be identified as the presence of the transgenic maize event LP026-2.

The nucleic acid sequence provided by the present invention may be at least 11 or more continuous polynucleotides (a first nucleic acid sequence) of any portion of the transgene insertion sequence in the SEQ ID NO:3 or a complementary sequence thereof, or at least 11 or more continuous polynucleotides of any portion of the 5′ flanking maize genomic DNA region in the SEQ ID NO:3 or a complementary sequence thereof (a second nucleic acid sequence). The nucleic acid sequence may further be homologous to or complementary to a portion of the SEQ ID NO:3 containing the complete SEQ ID NO:1. When the first nucleic acid sequence and the second nucleic acid sequence are used together, these nucleic acid sequences include a DNA primer pair in a DNA amplification method for generating amplification products. When the amplification product generated in the DNA amplification method by using a DNA primer pair is an amplification product including SEQ ID NO: 1, the presence of transgenic maize event LP026-2 or a progeny thereof can be diagnosed. It is well known to a person skilled in the art that the first and second nucleic acid sequences need not only be composed of DNA, but may also include RNA, a mixture of DNA and RNA, or a combination of DNA, RNA or other nucleotides or analogues thereof that are not used as templates for one or more polymerases. In addition, the probe or primer in the present invention should be at least about 11, 12, 13, 14, 15, 16, 17, 18, 19, 20, 21 or 22 continuous nucleotides in length, which can be selected from the nucleotides described in SEQ ID NO:1, SEQ ID NO:2, SEQ ID NO:3, SEQ ID NO:4 and SEQ ID NO:5. When selected from the nucleotides shown in SEQ ID NO:3, SEQ ID NO:4 and SEQ ID NO:5, the probe and primer may be continuous nucleotides with a length of about 17 to 50 or more. The SEQ ID NO:3 or a complementary sequence thereof is a sequence with a length of 1495 nucleotides located near the insertion junction site at the 5′ end of the insertion sequence in the transgenic maize event LP026-2, the SEQ ID NO:3 or a complementary sequence thereof consists of a maize flanking genomic DNA sequence of 995 nucleotides (nucleotides 1st to 995^{th} of SEQ ID NO: 3), a DNA sequence of pLP026 construct of 383 nucleotides (nucleotides 996^{th} to 1378^{th} of SEQ ID NO:3) and a 3′-terminal DNA sequence of a Nos terminator with 117 nucleotides (nucleotides 1379^{th} to 1495^{th} of SEQ ID NO:3), and the inclusion of the SEQ ID NO:3 or a complementary sequence thereof can be identified as the presence of the transgenic maize event LP026-2.

The nucleic acid sequence may be at least 11 or more continuous polynucleotides (a third nucleic acid sequence) of any portion of the transgene insertion sequence in the SEQ ID NO: 4 or a complementary sequence thereof, or at least 11 or more continuous nucleotides of any portion of the 3′-flanking maize genomic DNA region in the SEQ ID NO:4 or a complementary sequence thereof (a fourth nucleic acid sequence). The nucleic acid sequence may further be homologous to or complementary to a portion of the SEQ ID NO:4 containing the complete SEQ ID NO:2. When the third nucleic acid sequence and the fourth nucleic acid sequence are used together, these nucleic acid sequences include a DNA primer pair in the DNA amplification method for generating amplification products. When the amplification product generated in the DNA amplification method by using the DNA primer pair is an amplification product including SEQ ID NO:2, the presence of transgenic maize event LP026-2 or its progeny can be diagnosed. The SEQ ID NO:4 or a complementary sequence thereof is a sequence with a length of 590 nucleotides near the insertion junction site at the 3′ end of the insertion sequence in the transgenic maize event LP026-2, and the SEQ ID NO:4 or a complementary sequence thereof consists of a 53-nucleotide tNos (nopaline synthase) transcription terminator sequence (nucleotides 1^{st} to 53^{rd} of SEQ ID NO:4), a 201-nucleotide DNA sequence of a pLP026 construct (nucleotides 54^{th} to 254^{th} of SEQ ID NO:4) and a 336-nucleotide genomic DNA sequence flanking the maize integration site (nucleotides 255^{th} to 590^{th} of SEQ ID NO:4), and the inclusion of the SEQ ID NO:4 or a complementary sequence thereof can be identified as the presence of the transgenic maize event LP026-2.

The SEQ ID NO:5 or a complementary sequence thereof is a sequence with a length of 17,487 nucleotides characterizing the transgenic maize event LP026-2, which specifically contains the genome and genetic elements shown in Table 1. The inclusion of the SEQ ID NO: 5 or a complementary sequence thereof can be identified as the presence of the transgenic maize event LP026-2.

TABLE 1
Genome and genetic elements contained in SEQ ID NO: 5

| | | |
|---|---|---|
| genetic elements | Length | Location on SEQ ID NO: 5 |

| | | | |
|---|---|---|---|
| 5′ Genome | 995 | bp | 1-995 |
| RB | 383 | bp | 996-1378 |
| Nos | 253 | bp | 1379-1631 |
| Cry2Ab | 1905 | bp | 1661-3565 |
| ZmCTP | 415 | bp | 3566-3980 |
| ZmHSP70 | 806 | bp | 3981-4786 |
| pFMV | 595 | bp | 4792-5386 |
| ORF25PolyA | 713 | bp | 5393-6105 |
| Cry1Fa | 1818 | bp | 6112-7929 |
| pZmUbi | 1993 | bp | 7936-9928 |
| In2 | 344 | bp | 9973-10316 |
| Cry1Ab | 2457 | bp | 10324-12780 |
| iOsAct1 | 484 | bp | 12787-13270 |
| WtCab | 61 | bp | 13282-13342 |
| p35s | 322 | bp | 13355-13676 |
| pOsAct1 | 1411 | bp | 13685-15095 |
| AtCTP | 228 | bp | 15096-15323 |
| EPSPS | 1368 | bp | 15324-16691 |
| Nos | 253 | bp | 16698-16950 |
| LB | 201 | bp | 16951-17151 |
| 3′ Genome | 336 | bp | 17152-17487 |

The nucleic acid sequence or a complementary sequence thereof can be used in a DNA amplification method to generate an amplification product, and the presence of transgenic maize event LP026-2 or its progeny in a biological sample can be diagnosed through the detection of the amplification product; the nucleic acid sequence or a complementary sequence thereof can be used in a nucleotide detection method to detect the presence of transgenic maize event LP026-2 or its progeny in a biological sample.

The present invention provides a pair of DNA primers, which comprises a first primer and a second primer, wherein the first primer and the second primer each comprise a fragment of SEQ ID NO:5 or a complementary sequence thereof, and when used in an amplification reaction together with DNA containing maize event LP026-2, an amplification product is generated for the detection of maize event LP026-2 in a biological sample.

In some embodiments, the first primer is selected from SEQ ID NO:1 or a complementary sequence thereof, SEQ ID NO:8 or SEQ ID NO: 10; the second primer is selected from SEQ ID NO:2 or a complementary sequence thereof, SEQ ID NO:11 or SEQ ID NO: 14.

In some embodiments of the present invention, the amplification product comprises at least 11 continuous nucleotides in SEQ ID NO:3 or a complementary sequence thereof, or at least 11 continuous nucleotides in SEQ ID NO:4 or a complementary sequence thereof.

Further, the amplification product comprises the 1^{st} to 11^{th} or 12^{th} to 22^{nd} continuous nucleotides in SEQ ID NO:1 or a complementary sequence thereof, or the 1^{st} to 11^{th} or 12^{th} to 22^{nd} continuous nucleotides in SEQ ID NO:2 or a complementary sequence thereof.

Still further, the amplification product comprises SEQ ID NO:1 or a complementary sequence thereof, SEQ ID NO:2 or a complementary sequence thereof, SEQ ID NO:6 or a complementary sequence thereof, or SEQ ID NO:7 or a complementary sequence thereof.

In the above technical solutions, the primer comprises at least one of the nucleic acid sequences. Specifically, the primer comprises a first primer and a second primer, wherein the first primer is selected from SEQ ID NO:1 or a complementary sequence thereof, SEQ ID NO: 8 or SEQ ID NO:12, and the second primer is selected from SEQ ID NO:9 or SEQ ID NO: 13; or the first primer is selected from SEQ ID NO:2 or a complementary sequence thereof, SEQ ID NO:10 or SEQ ID NO:15, and the second primer is selected from SEQ ID NO: 11 or SEQ ID NO:14.

The present invention also provides a DNA probe, which comprises a fragment of SEQ ID NO:5 or a complementary sequence thereof, which hybridizes with a DNA molecule containing a nucleic acid sequence selected from the sequences consisting of SEQ ID NOs: 1-7 or the complementary sequences thereof under strict hybridization conditions and does not hybridize with a DNA molecule that does not contain a nucleic acid sequence selected from the sequences consisting of SEQ ID NOs: 1-7 or complementary sequences thereof under strict hybridization conditions.

In some embodiments, the DNA probe comprises a sequence selected from the group consisting of SEQ ID NO:1 or a complementary sequence thereof, SEQ ID NO:2 or a complementary sequence thereof, SEQ ID NO:6 or a complementary sequence thereof and SEQ ID NO:7 or a complementary sequence thereof.

In some embodiments, the DNA probe is labeled with a fluorophore.

In some embodiments, the probe comprises at least 11 continuous nucleotides in SEQ ID NO: 3 or a complementary sequence thereof, or at least 11 continuous nucleotides in SEQ ID NO: 4 or a complementary sequence thereof; further, the probe comprises the 1^{st} to 11^{th} or 12^{th} to 22^{nd} continuous nucleotides in SEQ ID NO:1 or a complementary sequence thereof, or the 1^{st} 11^{th} or 12^{th} to 22^{nd} continuous nucleotides in SEQ ID NO:2 or a complementary sequence thereof.

The present invention also provides a marker nucleic acid molecule, which comprises a fragment of SEQ ID NO:5 or a complementary sequence thereof, which hybridizes with a DNA molecule containing a nucleic acid sequence selected from the sequences consisting of SEQ ID NOs: 1-7 or complementary sequences thereof under strict hybridization conditions and does not hybridize with a DNA molecule that does not contain a nucleic acid sequence selected from the sequences consisting of SEQ ID NOs: 1-7 or a complementary sequences thereof under strict hybridization conditions.

In some embodiments, the marker nucleic acid molecule comprises a sequence selected from the group consisting of SEQ ID NO: 1 or a complementary sequence thereof, SEQ ID NO:2 or a complementary sequence thereof, SEQ ID NO:6 or a complementary sequence thereof and SEQ ID NO:7 or a complementary sequence thereof.

In one embodiment, the marker nucleic acid molecule comprises at least 11 continuous nucleotides in SEQ ID NO:3 or a complementary sequence thereof, or at least 11 continuous nucleotides in SEQ ID NO:4 or a complementary sequence thereof;

In some embodiments, the marker of nucleic acid molecule comprises continuous nucleotides at positions 1^{st} to 11^{th} or 12^{th} to 22^{nd} in SEQ ID NO:1 or a complementary sequence thereof, or continuous nucleotides at positions 1^{st} to 11^{th} or 12^{th} to 22^{nd} in SEQ ID NO: 2 or a complementary sequence thereof.

Further, the present invention provides a method for detecting a DNA containing transgenic maize event LP026-2 in a sample, comprising:
(1) contacting a sample to be detected with the pair of DNA primers in a nucleic acid amplification reaction;
(2) carrying out the nucleic acid amplification reaction; and
(3) detecting the amplified product;
wherein the inclusion of a nucleic acid sequence selected from the group consisting of SEQ ID NOs: 1-7 and the complementary sequences thereof indicates the existence of a DNA containing transgenic maize event LP026-2 in the detected sample.

The present invention also provides a method for detecting a DNA containing transgenic maize event LP026-2 in a sample, comprising:
(1) contacting a sample to be detected with the DNA probe and/or the marker nucleic acid molecule;
(2) hybridizing the sample to be detected with the probe and/or the marker nucleic acid molecule under strict hybridizing conditions; and
(3) detecting the hybridization of the sample to be detected to the probe and/or the marker nucleic acid molecule.

The strict condition may be hybridization in 6×SSC (sodium citrate) and 0.5% SDS (sodium dodecyl sulfate) solution at 65° C., followed by washing the membrane twice, once with 2×SSC, 0.1% SDS and once with 1×SSC and 0.1% SDS.

Wherein, the hybridization of the sample to be detected and the marker nucleic acid molecule is detected, and then the insect resistance and/or herbicide tolerance are determined to be genetically linked with the marker nucleic acid molecule through marker-assisted breeding analysis.

The present invention also provides a DNA detection kit, comprising a pair of DNA primers for creating an amplicon for detecting the transgenic maize event LP026-2, a probe specific for SEQ ID NOs: 1-7 or a marker nucleic acid molecule specific for SEQ ID NOs: 1-7. Specifically, the detection kit comprises the probe, the pair of primers or the marker nucleic acid molecule as described herein.

In some embodiments, the present invention provides a DNA detection kit, comprising at least one DNA molecule, which comprises at least 11 continuous nucleotides in a homologous sequence of SEQ ID NO:3 or a complementary sequence thereof, or at least 11 continuous nucleotides in a homologous sequence of SEQ ID NO:4 or a complementary sequence thereof, which can be used as a DNA primer or probe that is specific for the transgenic maize event LP026-2 or its progeny.

Further, the DNA molecule comprises continuous nucleotides at positions 1-11 or positions 12-22 in the sequence of SEQ ID NO:1 or a complementary sequence thereof, or continuous nucleotides at positions 1-11 or positions 12-22 in the sequence of SEQ ID NO:2 or a complementary sequence thereof.

Still further, the DNA molecule comprises a homologous sequence of SEQ ID NO: 1 or a complementary sequence thereof, a homologous sequence of SEQ ID NO:2 or a complementary sequence thereof, a homologous sequence of SEQ ID NO:6 or a complementary sequence thereof or a homologous sequence of SEQ ID NO:7 or a complementary sequence thereof. In order to achieve the above purposes, the present invention also provides a plant cell, which comprises a nucleic acid sequence encoding insect-resistant proteins Cry1Ab, Cry2Ab and Cry1Fa, and a nucleic acid sequence encoding glyphosate herbicide-resistant EPSPS protein and a nucleic acid sequence of a specific region, wherein the nucleic acid sequence of a specific region includes the sequences shown in SEQ ID NO: 1, SEQ ID NO:2, SEQ ID NO:6 or SEQ ID NO:7.

The sequences provided by the present invention include the sequences listed in Table 2 below:

TABLE 2
Relevant sequences of the present invention

| SEQ ID NO: | Description |
|---|---|
| 1 | RB-terminal junction-spanning sequence (containing partial T-DNA RB-terminal |
| | sequence and genomic sequence, about 22 bp) |
| 2 | LB-terminal junction-spanning sequence (containing partial T-DNA LB-terminal |
| | sequence and genomic sequence, about 22 bp) |
| 3 | Nucleotide sequence of the insertion sequence in which the 5′ end is located near |
| | the insertion junction site, which is the RB-terminal for T-DNA (containing about |
| | 1 kb of genomic sequence, and about 500 bp of T-DNA) |
| 4 | Nucleotide sequence of the insertion sequence in which the 3′ end is located near |
| | the insertion junction site, which is the the LB-terminal for T-DNA (contains about |
| | 400 bp of genomic sequence and about 300 bp of T-DNA) |
| 5 | T-DNA full-length sequence (containing genomic sequence extending ~400 bp at |
| | the LB-terminal and ~1 kb at the RB-terminal) |
| 6 | Sequence located within SEQ ID NO: 3, LP026 T-DNA sequence |
| 7 | Sequence located within SEQ ID NO: 4, LP026 T-DNA sequence |
| 8 | Primer 11, the first primer for amplifying SEQ ID NO: 3 |
| 9 | Primer 12, the second primer for amplifying SEQ ID NO: 3 |
| 10 | Primer 13, the first primer for amplifying SEQ ID NO: 4 |
| 11 | Primer 14, the second primer for amplifying SEQ ID NO: 4 |
| 12 | Primer 15, a primer for the 5′ flanking genome |
| 13 | Primer 16, a primer for T-DNA paired with SEQ ID NO: 12 |
| 14 | Primer 17, a primer for 3′ flanking genome |
| 15 | Primer 18, a primer for T-DNA paired with SEQ ID NO: 14 |
| 16 | Primer 1 for Taqman detection of Cry1Ab |
| 17 | Primer 2 for Taqman detection of Cry1Ab |
| 18 | Probe 1 for Taqman detection of Cry1Ab |
| 19 | Primer 3 for Taqman detection of Cry2Ab |
| 20 | Primer 4 for Taqman detection of Cry2Ab |
| 21 | Probe 2 for Taqman detection of Cry2Ab |
| 22 | Primer 5 for Taqman detection of Cry1Fa |
| 23 | Primer 6 for Taqman detection of Cry1Fa |
| 24 | Probe 3 for Taqman detection of Cry1Fa |
| 25 | Primer 7 for Taqman detection of EPSPS |
| 26 | Primer 8 for Taqman detection of EPSPS |
| 27 | Probe 4 for Taqman detection of EPSPS |
| 28 | A first primer 9 for endogenous gene zSSIIb of maize |
| 29 | A second primer 10 for endogenous gene zSSIIb of maize |
| 30 | Probe for Cry1Ab in Southern hybridization assay |
| 31 | Probe for Cry2Ab in Southern hybridization assay |
| 32 | Probe for Cry1Fa in Southern hybridization assay |
| 33 | Probe for EPSPS in Southern hybridization assay |
| 34 | Primer for T-DNA in the same orientation as SEQ ID NO: 13 |
| 35 | Primer for T-DNA in the opposite orientation as SEQ ID NO: 13 |
| 36 | Primer for T-DNA in the opposite orientation as SEQ ID NO: 13 |
| 37 | Primer for T-DNA in the same orientation as SEQ ID NO: 15 |
| 38 | Primer for T-DNA in the opposite orientation as SEQ ID NO: 15 |
| 39 | Primer for T-DNA in the opposite orientation as SEQ ID NO: 15 |

The present invention also provides a method for protecting maize plants from insects, comprising: providing at least one transgenic maize plant cell containing transgenic maize event LP026-2 in the diet of a target insect; the transgenic maize plant comprises, sequentially, SEQ ID NO:1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome, and a target insect that has ingested the transgenic maize plant cells is inhibited from further ingesting the maize plant.

The present invention also provides a method for protecting maize plants from damage caused by an herbicide, wherein at least one transgenic maize plant containing transgenic maize event LP026-2 is grown. The transgenic maize plant comprises, sequentially, SEQ ID NO: 1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome. In some embodiments, the method comprising applying an effective dose of glyphosate herbicide to the field where at least one transgenic maize plant is grown, wherein the transgenic maize plant comprises, sequentially, SEQ ID NO: 1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome.

The present invention also provides a method for controlling weeds in a field where maize plants are grown, comprising applying an effective dose of glyphosate herbicide to the field where at least one transgenic maize plant is grown, wherein the transgenic maize plant comprises, sequentially, SEQ ID NO: 1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome.

The present invention also provides a method for cultivating maize plants with insect resistance, comprising: growing at least one maize seed comprising the nucleic acid sequence of transgenic maize event LP026-2;
growing the maize seed into a maize plant;

Invading the maize plant with a target insect and/or spraying the maize plant with an effective dose of glyphosate herbicide, and harvesting the plants with reduced plant damage as compared to other plants without the transgenic maize event LP026-2.

In some embodiments, the present invention provides a method for cultivating maize plants that are resistant to insects and tolerant to glyphosate herbicides, comprising:
growing at least one maize seed comprising the nucleic acid sequence of transgenic maize event LP026-2;
growing the maize seed into a maize plant;
spraying the maize plant with an effective dose of glyphosate herbicide, and harvesting the plants with reduced plant damage as compared to other plants without the transgenic maize event LP026-2, and the plants with reduced plant damage are also resistant to damage from insect feeding.

In some embodiments, the present invention also provides a method for producing maize plants resistant to insects, comprising introducing transgenic maize event LP026-2 into the genome of the maize plants, and selecting maize plants with reduced plant damage from insect feeding. In some embodiments, the method comprises: sexually hybridizing a first parent maize plant with insect-resistant transgenic maize event LP026-2 to a second parent maize plant lacking insect resistance, thereby generating a large number of progeny plants; invading the progeny plants with target insects; selecting the progeny plants with reduced plant damage compared with other plants without transgenic maize event LP026-2.

In some embodiments, the present invention also provides a method for producing a maize plant with tolerance to glyphosate herbicide, comprising introducing transgenic maize event LP026-2 into the genome of the maize plant, and selecting a maize plant with tolerance to glyphosate. In some embodiments, the method comprises: sexually hybridizing a first parent maize plant with glyphosate herbicide-tolerant transgenic maize event LP026-2 to a second parent maize plant lacking tolerance to glyphosate herbicide, thereby generating a large number of progeny plants; treating the progeny plants with glyphosate herbicide; selecting the progeny plants tolerant to glyphosate.

In some embodiments, the present invention also provides a method for producing maize plants that are resistant to insects and tolerant to glyphosate herbicide application, comprising introducing transgenic maize event LP026-2 into the genome of the maize plants, and selecting maize plants that are resistant to glyphosate and have insect resistance. In some embodiments, the method comprises sexually hybridizing a first parent maize plant with transgenic maize event LP026-2 having tolerance to glyphosate herbicide and insect resistance to a second parent maize plant lacking tolerance to glyphosate herbicide and/or insect resistance, thereby generating a large number of progeny plants; treating the progeny plants with glyphosate; selecting the progeny plants tolerant to glyphosate, and the progeny plants that are tolerant to glyphosate are also resistant to damage from insect ingestion.

The present invention also provides a composition generated from transgenic maize event LP026-2, which is maize powder, maize flour, maize oil, maize car silk or maize starch. In some embodiments, the composition may be agricultural products or commodities such as maize powder, maize flour, maize oil, maize starch, maize gluten, maize cake, cosmetics or fillers. If a sufficient expression level is detected in the composition, the composition is expected to contain a nucleic acid sequence capable of diagnosing the transgenic maize event LP026-2 material in the composition. Specifically, the compositions include, but are not limited to, maize oil, maize meal, maize flour, maize gluten, maize cake, maize starch and any other food that will be used as a food source for animals to consume, or otherwise used as a bulking agent or a component in a cosmetic composition for cosmetic purposes, and the like.

The detection method and/or kit based on the probe or the pair of primers of the present invention may be used to detect the nucleic acid sequence of the transgenic maize event LP026-2 in a biological sample such as that shown in SEQ ID NO:1 or SEQ ID NO:2, wherein the probe sequence or the primer sequence is selected from the group consisting of sequences shown in, e.g., SEQ ID NO:1, SEQ ID NO:2, SEQ ID NO:3, SEQ ID NO:4 and SEQ ID NO:5 to diagnose the transgenic maize event LP026-2.

In summary, the transgenic maize event LP026-2 of the present invention has a dual characteristics of insect resistance and herbicide tolerance, and has the following advantages: 1) protection from economic losses due to Lepidoptera pests (such as Ostrinia furnacalis, Mythimna separata, Spodoptera frugiperda, Helicoverpa armigera, Agrotis ipsilon, Conogethes punctiferalis and the likes, which are major pests in maize-growing areas); 2) the ability to apply glyphosate-containing agricultural herbicides to maize crops for broad-spectrum weed control; and 3) no reduction in maize yields. Specifically, the event LP026-2 of the present invention has a high resistance level to target pests, which can make the mortality rate of pests as high as 100% and protect plants to make their damage rate as low as 0%. High tolerance to glyphosate herbicide, which can protect plants and make their damage rate as low as 0%; moreover, the agronomic characters of plants containing this event are excellent, and the yield percentage can be as high as 100%. In addition, the genes encoding insect resistance and glyphosate tolerance are linked to the same DNA segment and exist at a single locus in the genome of transgenic maize event LP026-2, which improves the breeding efficiency and enables the use of molecular markers to track the transgene insertion fragments in breeding populations and their progeny. At the same time, the primer or probe sequence provided in the detection method of the present invention can generate amplification products identified as transgenic maize event LP026-2 or its progeny, and can quickly, accurately and stably identify the plant materials originating from transgenic maize event LP026-2.

### Terms

The following definitions and methods will better define the present invention and guide a person skilled in the art to implement the present invention. Unless otherwise specified, the terms are to be understood in accordance with the conventional usage of a person skilled in the art.

The “maize” used herein refers to Zea mays, and includes all plant varieties that can be mated with maize, including wild maize species.

The “including” means “including but not limited to”. The “processed product” means a product, such as composition, obtained by processing raw materials such as plants and seeds.

The term “plant” includes whole plants, plant cells, plant organs, plant protoplasts, plant cell tissue cultures from which plants can regenerate, plant calli, plant clumps and whole plant cells in plants or plant parts, wherein the plant parts, are, for example embryos, pollen, ovules, seeds, leaves, flowers, branches, fruits, stems, roots, root tips, anthers and the like. It should be understood that the parts of transgenic plants within the scope of the present invention include, but are not limited to, plant cells, protoplasts, tissues, calli, embryos, flowers, stems, fruits, leaves and roots, which are derived from transgenic plants transformed with the DNA molecules of the present invention in advance and therefore at least partially composed of transgenic cells or their progeny.

The term “gene” refers to a nucleic acid fragment that expresses a specific protein, including a regulatory sequence that precedes the coding sequence (5′ non-coding sequence) and a regulatory sequence that follows the coding sequence (3′ non-coding sequence). A “natural gene” refers to a gene naturally found with its own regulatory sequence. A “chimeric gene” refers to any gene that is not a natural gene, which contains regulatory and coding sequences that are not found naturally. An “endogenous gene” refers to a natural gene which is located in its natural position in the genome of an organism. An “exogenous gene” is a foreign gene that is present in the genome of an organism and was not originally present, and also refers to the gene introduced into the recipient cell through the transgenic step. Exogenous genes may include natural genes or chimeric genes inserted into non-natural organisms. A “transgene” is a gene that has been introduced into the genome through transformation procedures. The site where recombinant DNA has been inserted in plant genome may be referred to as the “insertion site” or “target site”.

A “flanking DNA” may include a genome that is naturally present in an organism such as a plant or an exogenous (heterologous) DNA introduced through a transformation process, such as a fragment associated with a transformation event. Therefore, flanking DNA may include a combination of natural and exogenous DNA. In the present invention, “flanking region” or “flanking sequence” or “genomic boundary region” or “genomic boundary sequence” means a sequence of at least 3, 5, 10, 11, 15, 20, 50, 100, 200, 300, 400, 1000, 1500, 2000, 2500, 5000 or more base pairs, which is located directly upstream or downstream of and adjacent to the initial exogenously inserted DNA molecule. When the flanking region is located downstream, it may also be referred to as a “left boundary flanking sequence” or “3′ flanking sequence” or “3′ genome boundary region” or “genome 3′ boundary sequence” and so on. When the flanking region is located upstream, it may also be referred to as a “right boundary flanking sequence” or “5′ flanking sequence” or “5′ genome boundary region” or “genome 5′ boundary sequence” and so on.

Transformation procedures that cause random integration of exogenous DNA will lead to transformants with different flanking regions, the different flanking regions are specifically contained by each transformant. When recombinant DNA is introduced into plants by conventional hybridization, the flanking region thereof usually does not change. Transformants will also contain unique junctions between segments of heterologous insert DNA and genomic DNA or between two segments of genomic DNA or between two segments of heterologous DNA. A “junction” is a point where two specific DNA fragments are joined. For example, a junction exists where an insert DNA joins to the flanking DNA. A junction point also exists in a transformed organism, in which two DNA fragments are joined together in a way modified from that found in natural organisms. A “junction DNA” refers to DNA containing a junction point.

The present invention provides a transgenic maize event known as LP026-2 and its progeny, wherein the transgenic maize event LP026-2 is a maize plant LP026-2, which comprises the plant and seed of the transgenic maize event LP026-2 and the plant cells thereof or reproducible portions thereof, the plant portions of the plant and seed including, but not limited to, the cells, pollen, ovules, flowers, buds, roots, stems, ear silks, inflorescences, ear tassels, leaves and products from the maize plant LP026-2, such as maize powder, maize flour, maize oil, maize syrup, maize ear silk, maize starch, and biomass left in the field of the maize fields.

The transgenic maize event LP026-2 of the present invention contains a DNA construct, and when it is expressed in a plant cell, the transgenic maize event LP026-2 gains resistance to insects and tolerance to glyphosate herbicide.

In some embodiments of the present invention, the DNA construct comprises four expression cassettes connected in series, the first expression cassette comprises a suitable promoter for expression in plants and a suitable polyadenylation signal sequence, wherein the promoter is operably linked to the nucleic acid sequence of insect-resistant Cry2Ab protein (cCry2Ab) of Bacillus thuringiensis, and the Cry2Ab protein has Lepidoptera insect resistance; the second expression cassette contains a suitable promoter for expression in plants and a suitable polyadenylation signal sequence, wherein the promoter is operably linked to the nucleic acid sequence of insect-resistant Cry1Fa protein (cCry1Fa) of Bacillus thuringiensis, and the Cry1Fa has Lepidoptera insect resistance; and the third expression cassette contains a suitable promoter for expression in plants and a suitable polyadenylation signal sequence, wherein the promoter is operably connected with the nucleic acid sequence of Cry1 Ab protein, and the nucleic acid sequence of Cry1Ab protein is mainly resistant to Lepidoptera insects. The fourth expression cassette contains a suitable promoter for expression in plants and a suitable polyadenylation signal sequence, wherein the promoter is operably linked with a gene encoding 5-enolpyruvylshikimate-3-phosphate synthase (EPSPS), and the nucleic acid sequence of the EPSPS protein is tolerant to glyphosate herbicides. Further, the promoter may be a suitable promoter isolated from plants, including constitutive, inducible and/or tissue-specific promoters, the suitable promoter includes, but is not limited to, a cauliflower mosaic virus (CaMV) 35S promoter, a Figwort mosaic virus (FMV) 35S promoter, an Ubiquitin promoter, an Actin promoter, an Agrobacterium tumefaciens nopaline synthase (NOS) promoter, an octopine synthase (OCS) promoter, a Cestrum yellow leaf curling virus promoter, a potato tuber storage protein (Patatin) promoter, a ribulose-1,5-diphosphate carboxylase/oxygenase (RuBisCO) promoter, a glutathione S-transferase (GST) promoter, an E9 promoter, a GOS promoter, an alcA/alcR promoter, an Agrobacterium rhizogenes RolD promoter and an Arabidopsis thaliana Suc2 promoter. The polyadenylation signal sequence may be a suitable polyadenylation signal sequence that functions in plants, and the suitable polyadenylation signal sequence includes, but is not limited to, a polyadenylation signal sequence derived from nopaline synthase (NOS) gene of Agrobacterium tumefaciens, a polyadenylation signal sequence derived from cauliflower mosaic virus (CaMV) 35S terminator and protease inhibitor II (PIN II) gene, and a polyadenylation signal sequence derived from α-tubulin gene.

In addition, the expression cassette may also include other genetic elements, the genetic elements include, but are not limited to, enhancers and signal peptide/transport peptide nucleic acid coding sequences. The enhancer can enhance the expression level of the gene, and the enhancer includes, but is not limited to, a tobacco etching virus (TEV) translation activator, a CaMV35S enhancer and a FMV35S enhancer. The signal peptide/transport peptide can guide Cry1Ab protein and/or EPSPS protein to transport to outside or specific organelles or compartments inside cells, for example, targeting chloroplasts using sequences encoding chloroplast transport peptides or targeting the endoplasmic reticulum using ‘KDEL’ retention sequences.

The Cry1Ab, Cry2Ab and Cry1Fa genes can be isolated from Bacillus thuringiensis (Bt for short), and the nucleic acid sequences of Cry1Ab, Cry2Ab and Cry1Fa genes can be changed by optimizing codons or in other ways, so as to increase the stability of transcripts and availability in transformed cells.

In some embodiments of the present invention, a maize cell, seed or plant containing transgenic maize event LP026-2 comprises, sequentially, SEQ ID NO:1, the nucleic acid sequence at positions 1007-17140 of SEQ ID NO:5 and SEQ ID NO: 2 in its genome, or alternatively, comprises SEQ ID NO:5 in its genome.

The scientific name of “Lepidoptera”, includes moths and butterflies, and is an order with the largest number of agricultural and forestry pests, such as Ostrinia furnacalis, Helicoverpa armigera, Mythimna separata, Spodoptera frugiperda, Athetis lepigone, Conogethes punctiferalis and the likes.

The 5-enolpyruvylshikimate-3-phosphate synthase (EPSPS) gene may be isolated from Agrobacterium tumefaciens sp. CP4 strain, and the polynucleotide of the gene encoding the EPSPS may be changed by optimizing codons or in other ways, so as to achieve the purpose of increasing the stability of transcripts and availability in transformed cells. The 5-enolpyruvylshikimate-3-phosphate synthase (EPSPS) gene may also be used as a selective marker gene.

The “glyphosate” refers to N-phosphonomethyl glycine and its salts, and the treatment with “glyphosate herbicide” refers to the treatment with any herbicide preparation containing glyphosate. The selection of the utilization rate of a glyphosate formulation to achieve an effective biological dose is not beyond the skill of ordinary agrotechnicians. Treatment of a field comprising plant material derived from transgenic maize event LP026-2 with any of the glyphosate-containing herbicide formulations will control weed growth in the field and will not affect the growth or yield of plant material derived from transgenic maize event LP026-2.

The DNA construct is introduced into a plant by transformation methods, including but not limited to an Agrobacterium-mediated transformation method, a gene gun transformation method and a pollen tube pathway transformation method.

The Agrobacterium-mediated transformation method is a commonly used method for plant transformation. The exogenous DNA to be introduced into a plant is cloned into the shared sequences between the left and right borders of the vector, i.e. the T-DNA region. The vector is transformed into an Agrobacterium cell, subsequently, the Agrobacterium cell is used to infect a plant tissue, and the T-DNA region of the vector containing the exogenous DNA is inserted into the plant genome.

The gene gun transformation method is to bombard a plant cell with a vector containing exogenous DNA (particle-mediated biological bullet transformation).

The pollen tube pathway transformation method is to use the natural pollen tube channel (also known as pollen tube guiding tissue) formed by plant pollination to carry the exogenous DNA into embryo sac via the bead core channel.

After transformation, transgenic plants must be regenerated from the transformed plant tissue and the progeny with the exogenous DNA must be selected using suitable markers.

A DNA construct is an assembly of DNA molecules interconnected to each other that provide one or more expression cassettes. DNA constructs are specifically plasmids that are capable of self-replicating within bacterial cells and contain different restriction endonuclease sites for the introduction of DNA molecules that provide functional gene elements, namely promoters, introns, leader sequences, coding sequences, 3′ terminator regions and other sequences. The expression cassette contained in the DNA construct includes gene elements necessary to provide transcription of messenger RNA, and the expression cassette may be designed for expression in prokaryotic or eukaryotic cells. The expression cassette of the present invention is designed to be expressed, most specifically, in plant cells.

A transgenic “event” is obtained by transforming a plant cell with a heterologous DNA construct, that is, including the steps of insertion of the nucleic acid expression cassette containing at least one target gene into a plant genome by transgenic method to generate a plant population, regeneration of the plant population and selection of a particular plant characterized by insertion into a particular genomic locus. The term “event” refers to the original transformant including heterologous DNA and the progeny of the transformant. The term “event” also refers to the progeny resulting from a sexual hybridization between transformants and individuals of other varieties containing heterologous DNA, and the inserted DNA and flanking genomic DNA from parent transformant s are present at the same chromosomal position in the hybrid progeny even after repeated backcrosses with backcross parents. The term “event” also refers to a DNA sequence from an original transformant, the DNA sequence contains an inserted DNA and flanking genomic sequences closely adjacent to the inserted DNA, and the DNA sequence is expected to be transferred to a progeny, which is produced by sexual hybridization between a parent line containing the inserted DNA (e.g., the original transformant and its self-bred progeny) and a parent line that does not contain the inserted DNA, and the progeny receives the inserted DNA containing the target gene.

In the present invention, “recombination” refers to a form of DNA and/or protein and/or organism that is not normally found in nature and therefore produced by artificial intervention. Such artificial intervention may produce recombinant DNA molecules and/or recombinant plants. The “recombinant DNA molecule” is obtained by artificially combining two otherwise separated sequence segments, for example by chemical synthesis or by manipulating separated nucleic acid segments by genetic engineering techniques. The techniques for manipulating nucleic acids are well known.

The term “transgene” includes any cell, cell line, callus, tissue, plant part or plant, the genotype of which is changed due to the existence of heterologous nucleic acid, and the “transgene” includes the transgene originally changed in this way and the progeny individuals generated by the original transgene through sexual hybridization or asexual reproduction. In the present invention, the term “transgene” does not include genomic (chromosomal or extrachromosomal) changes by conventional plant breeding methods or naturally occurring events, such as random heterologous fertilization, non-recombinant viral infection, non-recombinant bacterial transformation, non-recombinant transposition or spontaneous mutation.

“Heterologous” in the present invention means that a first molecule is not usually found to combine with a second molecule in nature. For example, a molecule may originate from a first species and be inserted into the genome of a second species. Therefore, such molecule is heterologous to a host and artificially introduced into the genome of the host cell.

The cultivation of a transgenic maize event LP026-2 that is resistant to Lepidoptera insects and tolerant to glyphosate herbicide may be achieved by the following steps: firstly, sexually hybridizing a first parent maize plant to a second parent maize plant, thereby generating a variety of first generation progeny plants, wherein the first parent maize plants comprises maize plants cultivated from the transgenic maize event LP026-2 and its progeny, and the transgenic maize event LP026-2 and its progeny are obtained by transforming the expression cassette of the present invention with resistance to Lepidoptera insects and tolerance to glyphosate herbicide, and the second parent maize plant lacks resistance to Lepidoptera insects and/or tolerance to glyphosate herbicide; the progeny plants with resistance to the invasion of Lepidoptera insects and/or tolerance to glyphosate herbicide arc then selected, so that maize plants with resistance to Lepidoptera insects and tolerance to glyphosate herbicide may be cultivated. These steps may further include backcrossing the progeny plants with resistance to Lepidoptera insects and/or tolerance to glyphosate herbicide to a second parent maize plant or a third parent maize plant, and then selecting the progeny by infection with Lepidoptera insects, by application of glyphosate herbicide, or by identification of molecular markers associated with traits (e.g., DNA molecules containing junction sites identified at the 5′ end and 3′ end of insertion sequence in transgenic maize event LP026-2), thereby producing maize plants that are resistant to Lepidoptera insects and tolerant to glyphosate herbicide.

It should be further understood that two different transgenic plants can also cross to produce progeny containing two independent and separately added exogenous gene. Self-crossing of appropriate progeny may result in progeny plants that are homozygous for both added exogenous genes. Backcrosses to parental plants and heterozygous crosses to non-transgenic plants as described earlier are also to be expected, as is asexual reproduction.

The term “probe” is a segment of an isolated nucleic acid molecule to which conventional detectable markers or reporter molecules can be bound, such as a radioisotope, a ligand, chemiluminescence agent or enzyme. Such a probe is complementary to a strand of the target nucleic acid, and in the present invention, the probe is complementary to a DNA strand from the genome of transgenic maize event LP026-2, regardless of whether the genomic DNA is derived from transgenic maize event LP026-2 or seeds thereof or plants derived from transgenic maize event LP026-2 or seeds or extracts thereof. The probe of the present invention includes not only deoxyribonucleic acid or ribonucleic acid, but also polyamide and other probe materials that specifically bind to the target DNA sequence and can be used to detect the target DNA sequence.

The term “primer” is a segment of an isolated nucleic acid molecule, which is annealed to a complementary target DNA strand by nucleic acid hybridization, forms a hybrid between the primer and the target DNA strand, and then extends along the target DNA strand under the action of a polymerase (such as DNA polymerase). The pair of primers of the present invention relates to its application in target nucleic acid sequence amplification, for example, by polymerase chain reaction (PCR) or other conventional nucleic acid amplification methods.

Methods for designing and using primers and probes are well known in the art. DNA molecules containing the full length or fragments of SEQ ID NOs: 1-7 may be used as primers and probes for detecting maize event LP026-2, and may be easily designed by those skilled in the art using the sequences provided herein.

The length of the probe and primer is generally 11 polynucleotides or more, preferably 18 polynucleotides or more, more preferably 24 polynucleotides or more, and most preferably 30 polynucleotides or more. Such probes and primers hybridize specifically with the target sequence under highly strict hybridization conditions. Although probes that are different from the target DNA sequence and maintain the ability of hybridizing with the target DNA sequence may be designed by conventional methods, it is preferable that the probes and primers in the present invention have complete DNA sequence identity with the continuous nucleotides of the target sequence.

Primers and probes based on the flanking genomic DNA and insertion sequence of the present invention may be determined by a conventional method, for example, by isolating the corresponding DNA molecule from the plant material of transgenic maize event LP026-2 and determining the nucleic acid sequence of the DNA molecule. The DNA molecule contains transgene insertion sequence and flanking region of maize genome, and the fragment of the DNA molecule may be used as primer or probe.

The nucleic acid probe and primer of the present invention hybridize to the target DNA sequence under strict conditions. Any conventional nucleic acid hybridization or amplification method may be used to identify the DNA from transgenic maize event LP026-2 in a sample. Nucleic acid molecules or fragments thereof are capable of hybridizing specifically with other nucleic acid molecules under certain conditions. As used in the present invention, two nucleic acid molecules are said to be capable of specifically hybridizing with each other if they are capable of forming a reverse parallel double-stranded nucleic acid structure. If the two nucleic acid molecules show complete complementarity, one of them is said to be the “complement” of the other nucleic acid molecule. As used in the present invention, two nucleic acid molecules are said to show “full complementarity” when each nucleotide of one nucleic acid molecule is complementary to the corresponding nucleotide of the other nucleic acid molecule. Two nucleic acid molecules are said to be “minimally complementary” if they are capable of hybridizing to each other with sufficient stability so that they can anneal to each other under at least conventional “low strict” conditions. Similarly, two nucleic acid molecules are said to be “complementary” if they are capable of hybridizing to each other with sufficient stability so that they can anneal to each other under conventional “highly strict” conditions. Deviation from complete complementarity is permissible as long as this deviation does not completely prevent the two molecules from forming a double-stranded structure. In order to make a nucleic acid molecule as a primer or probe, it is only necessary to ensure that it is sufficiently complementary in sequence to allow for the formation of a stable double-stranded structure at the particular solvent and salt concentration employed.

As used in the present invention, a substantially homologous sequence is a segment of a nucleic acid molecule which is capable of hybridizing specifically with the complementary strand of a matching segment of another nucleic acid molecule under highly stringent conditions. Suitable strict conditions for promoting DNA hybridization, such as treating with 6.0×sodium chloride/sodium citrate (SSC) at about 45° C. and then washing with 2.0×SSC at 50° C., are well known to a person skilled in the art. For example, the salt concentration in the washing step may be selected from about 2.0×SSC, 50° C. under low strict conditions to about 0.2×SSC, 50° C. under highly strict conditions. In addition, the temperature condition in the washing step may be elevated from about 22° C. at room temperature under low strict conditions to about 65° C. under highly strict conditions. Both temperature conditions and salt concentration may be changed, or one of them may be kept unchanged while the other variable is changed. Specifically, a nucleic acid molecule of the present invention may react with one or more nucleic acid molecules in SEQ ID NO:1, SEQ ID NO:2, SEQ ID NO:3, SEQ ID NO:4, SEQ ID NO:5, SEQ ID NO:6 and SEQ ID NO:7 or their complementary sequences, or any fragment of the above-mentioned sequences under moderately strict conditions, for example, at about 2.0×SSC and about 65° C. More specifically, a nucleic acid molecule of the present invention specifically hybridizes with one or more nucleic acid molecules in SEQ ID NO:1, SEQ ID NO:2, SEQ ID NO:3, SEQ ID NO:4, SEQ ID NO:5, SEQ ID NO:6 and SEQ ID NO:7 or their complementary sequences, or any fragment of the above sequences under highly strict conditions. In the present invention, the preferred marker of nucleic acid molecule has SEQ ID NO:1, SEQ ID NO:2, SEQ ID NO:6 or SEQ ID NO:7 or the complementary sequences thereof, or any fragment of the above sequences. Another preferred marker of nucleic acid molecule of the present invention has 80% to 100% or 90% to 100% sequence identity with SEQ ID NO:1, SEQ ID NO:2, SEQ ID NO:6 or SEQ ID NO: 7 or their complementary sequences, or any fragment of the above sequences. SEQ ID NO: 1, SEQ ID NO:2, SEQ ID NO:6 and SEQ ID NO:7 may be used as markers in plant breeding methods to identify the progeny of genetic hybridization. Hybridization between the probe and the target DNA molecule may be detected by any method known to those skilled in the art, including but not limited to fluorescent labeling, radioactive labeling, antibody-based labeling and chemiluminescence labeling.

With regard to amplification of a target nucleic acid sequence using specific amplification primers, for example, by PCR, “strict conditions” refers to the conditions that only primers are allowed to hybridize to the target nucleic acid sequence in a DNA thermal amplification reaction, and a primer with a wild-type sequence (or a complementary sequence thereof) corresponding to the target nucleic acid sequence can bind to the target nucleic acid sequence, and preferably produces a unique amplification product, namely amplicon.

The term “specific binding (to a target sequence)” refers to that a probe or primer hybridizes only with the target sequence in a sample containing the target sequence under a strict hybridization condition.

As used in the present invention, “amplified DNA” “amplification product” or “amplicon” refers to a nucleic acid amplification product of a target nucleic acid sequence as part of a nucleic acid template. For example, in order to determine whether a maize plant is produced by sexual hybridization with the transgenic maize event LP026-2 of the present invention, or whether a maize sample collected from a field contains the transgenic maize event LP026-2, or whether a maize extract, such as maize meal, powder or oil, contains the transgenic maize event LP026-2, DNA extracted from a maize plant tissue sample or extract may be amplified by a nucleic acid amplification method using primer pairs to generate an amplicon to diagnose the existence of DNA for the transgenic maize event LP026-2. The pair of primers includes a first primer derived from a flanking sequence adjacent to the insertion site of the inserted exogenous DNA in the plant genome and a second primer derived from the inserted exogenous DNA. The amplicon has a certain length and sequence, and the sequence is also diagnostic for the transgenic maize event LP026-2. The length of the amplicon may range from the binding length of the primer pair plus one nucleotide base pair, preferably plus about fifty nucleotide base pairs, more preferably plus about two hundred and fifty nucleotide base pairs, most preferably plus about four hundred and fifty nucleotide base pairs or more.

Alternatively, the primer pair may be derived from the genomic sequences flanking the insertion DNA on both sides to generate an amplicon including the entire insertion nucleic acid sequence. One primer of the primer pair derived from the plant genomic sequence may be a certain distance away from the insertion DNA sequence, from one nucleotide base pair to about 20,000 nucleotide base pairs. The use of the term “amplicon” specifically excludes primer dimers formed in the thermal amplification reaction of DNA.

The nucleic acid amplification reaction may be realized by any nucleic acid amplification reaction method known in the art, including polymerase chain reaction (PCR). Various nucleic acid amplification methods are well known to those skilled in the art. The PCR amplification method has been developed to amplify 22 kb of genomic DNA and 42 kb of phage DNA. These methods, as well as other DNA amplification methods in the art can be used in the present invention. The inserted exogenous DNA sequence and flanking DNA sequence from transgenic maize event LP026-2 may be amplified by using the provided primer sequences, and then the PCR amplicon or cloned DNA is subjected to standard DNA sequencing.

The DNA detection kit based on DNA amplification method may contain DNA primer molecules, which specifically hybridize to the target DNA and amplify the diagnostic amplicon under appropriate reaction conditions. The kit may provide an agarose gel-based detection method or many methods known in the art for detecting diagnostic amplicon. A kit containing DNA primers homologous or complementary to any portion of the maize genome region of SEQ ID NO:3 or SEQ ID NO:4 and homologous or complementary to any portion of the transgene insertion region of SEQ ID NO:5 is provided by the present invention. Particularly, the primer pair useful in the DNA amplification method are identified as SEQ ID NO: 8 and SEQ ID NO:9, which amplify the diagnostic amplicon homologous to a portion of the 5′ transgene/genome region of the transgenic maize event LP026-2, wherein the amplicon includes SEQ ID NO:1. Other DNA molecules used as DNA primers may be selected from SEQ ID NO:5.

Amplicon produced by these methods may be detected by various techniques. One method is Genetic Bit Analysis, which designs a DNA oligonucleotide strand that spans the inserted DNA sequence and the adjacent flanking genomic DNA sequence. The oligonucleotide strand was immobilized in the microwell of a microplate. After PCR amplification of the target region (one primer was used in the insertion sequence and one primer was used in the adjacent flanking genomic sequence), the single-stranded PCR product could hybridize with the immobilized oligonucleotide strand, and it was used as a template for the single-base extension reaction, which used DNA polymerase and ddNTPs specifically labeled for the next expected base. The results may be obtained by methods like fluorescence or ELISA. The signal represents the presence of insertion/flanking sequences, which indicates that amplification, hybridization and single base extension reactions are successful.

Another method is Pyrosequencing. In this method, an oligonucleotide strand spanning the inserted DNA sequence and the adjacent genomic DNA binding site was designed. The oligonucleotide strand was hybridized to the single-stranded PCR product of the target region (one primer was used for the insertion sequence and one primer was used for the adjacent flanking genomic sequence), and then incubated with DNA polymerase, ATP, thiolase, luciferase, adenosine triphosphate bisphosphatase, adenosine-5′-phosphosulfate and fluorescein. The dNTPs were added separately to measure the generated optical signal. The optical signal represents the presence of insertion/flanking sequences, which indicates that amplification, hybridization, and single-base or multi-base extension reactions are successful.

The fluorescence polarization phenomenon described by Chen et al. (Genome Res. 9:492-498, 1999) is also a method that can be used to detect the amplicon of the present invention. Using this method, it is necessary to design an oligonucleotide strand that spans the inserted DNA sequence and the adjacent genomic DNA binding site. The oligonucleotide strand is hybridized to the single-stranded PCR product of the target region (one primer was used for the insertion sequence and one primer was used for the adjacent flanking genomic sequence), and then incubated with DNA polymerase and a fluorescent labeled ddNTPs. Single base extension result in the insertion of ddNTPs. This insertion can be measured using a fluorometer to measure the change in its polarization. The change of the polarization represents the existence of insertion/flanking sequences, which indicates that amplification, hybridization and single base extension reactions are successful.

Taqman is described as a method for detecting and quantitatively analyzing the existence of DNA sequences, which is described in detail in the instructions provided by the manufacturer. Now, a brief example is given as follows: designing a FRET oligonucleotide probe spanning the inserted DNA sequence and the adjacent flanking binding site of the genome. The FRET probe and PCR primers (one primer for the insertion sequence and one primer for the adjacent flanking genomic sequence) are subjected to cyclic reaction in the presence of thermostable polymerase and dNTPs. Hybridization of FRET probe leads to the splitting of fluorescence portion and quenching portion on FRET probe and the release of fluorescence portion. The generation of fluorescence signal represents the existence of insertion/flanking sequence, which indicates that amplification and hybridization are successful.

Based on the principle of hybridization, suitable techniques for detecting plant materials derived from transgenic maize event LP026-2 may also include Southern blot hybridization, Northern blot hybridization and in situ hybridization. In particular, the suitable technique includes incubating the probe and the sample, washing to remove unbound probes and detecting whether the probe has hybridized. The detection method depends on the type of label attached to the probe, for example, a radiolabeled probe can be detected by exposure and development of an X-ray film or an enzyme-labeled probe can be detected by achieving a color change through substrate conversion.

Tyangi et al. (Nat. Biotech. 14:303-308, 1996) introduced the application of molecular markers in sequence detection. Briefly described as follows, a FRET oligonucleotide probe spanning the inserted DNA sequence and the adjacent flanking binding site of the genome was designed. The unique structure of the FRET probe leads to it having a secondary structure, which can keep the fluorescence portion and quenching portion in a short distance. The FRET probe and PCR primers (one primer in the insertion sequence and one primer in the adjacent flanking genomic sequence) are subjected to cyclic reaction in the presence of thermostable polymerase and dNTPs. After successful PCR amplification, the hybridization between FRET probe and target sequence leads to the loss of the secondary structure of the probe, so that the fluorescence portion and quenching portion are separated in space and the fluorescence signal is generated. The generation of fluorescence signal represents the existence of insertion/flanking sequence, which indicates that amplification and hybridization are successful.

Other described methods, such as microfluidics, provide methods and devices for isolating and amplifying DNA samples. Photodyes are used to detect and determine specific DNA molecules. Nanotube devices that contain electronic sensors for detecting DNA molecules or nanobeads that bind to specific DNA molecules and are thus detectable are useful for detecting the DNA molecules of the present invention.

The composition described in the present invention and the methods described or known in the art of DNA detection may be used to develop DNA detection kits. The kit facilitates the identification of the presence of DNA of the transgenic maize event LP026-2 in a sample and can also be used to breed maize plants containing DNA of the transgenic maize event LP026-2. The kit may contain DNA primers or probes which are homologous to or complementary to at least a part of SEQ ID NO:1, 2, 3, 4 or 5, or other DNA primers or probes which are homologous to or complementary to DNA contained in transgenic genetic elements of the DNA, and these DNA sequences may be used for DNA amplification reactions or as probes in DNA hybridization methods.

The DNA structure at the site joining the transgene insertion sequence to the maize genome contained in the maize genome and illustrated in FIG. 1 and Table 1 includes a flanking genomic region of maize LP026-2 at the 5′ end of the transgene insertion sequence, a part of the insertion sequence from the right border (RB) region of Agrobacterium tumefaciens, and the first expression cassette is composed of the Figwort mosaic virus 35s promoter (prFMV), operatively linked to an intron of maize heat shock protein HSP70 gene (iZmHSP70), operably linked to a maize chloroplast transport peptide 2 (spZmCTP2), operably linked to an insect-resistant Cry2Ab protein of Bacillus thuringiensis (cCry2Ab), and operably linked to a transcription terminator of nopaline synthase (tNos); the second expression cassette is composed of tandem repeated promoters of maize ubiquitin gene Ubi containing an enhancer region (prZmUbi), operably linked to an insect-resistant gene Cry1Fa of Bacillus thuringiensis (cCry1Fa), and operably linked to a terminator ORF25PolyA from Agrobacterium tumefaciens pTi15955; the third expression cassette is composed of a cauliflower mosaic virus (CaMV) 35S promoter (pr35S), operably linked to the non-coding leading strand sequence at the 5′ end of the wheat chloroplast a/b binding protein (lWtCab), operably linked to the rice actin gene 1 intron (iOsAct1), operably linked to the insect-resistant Cry1Ab protein of Bacillus thuringiensis (cCry1Ab), and operably linked to the terminator of the benzenesulfonamide-inducible gene 2 (tin2); and the fourth expression cassette is composed of the rice actin 1 promoter (prOsAct1), operably linked to Arabidopsis chloroplast transporter peptide (spAtCTP2), operably linked to glyphosate-tolerant 5-enolpyruvylshikimate-3-phosphate synthase (cEPSPS) of Agrobacterium strain CP4, and operably linked to transcription terminator of nopaline synthase (tNos). A portion of the sequence from the left border region (LB) of Agrobacterium, as well as the flanking genomic region of maize plant LP026-2 located at the 3′ end of the transgene insertion sequence (SEQ ID NO: 5) is inserted into the sequence. In the DNA amplification method, the DNA molecule as a primer may be any portion of the transgene insertion sequence in the transgenic maize event LP026-2, or any portion of the DNA region of the flanking maize genome in the transgenic maize event LP026-2.

The transgenic maize event LP026-2 may be combined with other transgenic maize varieties, such as maize with herbicide (such as glufosinate, Decamba and the like) tolerance, or transgenic maize varieties with other insect (such as scarabs, white grubs and Monolepta hieroglyphica)-resistant genes. Various combinations of all these different transgenic events, bred together with the transgenic maize event LP026-2 of the present invention, may provide improved hybrid transgenic maize varieties resistant to various pests and tolerant to various herbicides. Compared with non-transgenic varieties and single-trait transgenic varieties, these varieties may show more excellent characteristics such as yield increase.

The present invention provides a transgenic maize event LP026-2, which is used for detecting the nucleic acid sequence of maize plants containing the event and a detection method thereof. The transgenic maize event LP026-2 is resistant to the feeding damage by Lepidoptera pests and tolerant to the phytotoxicity of agricultural herbicides containing glyphosate. The dual-trait maize plant expresses Cry1Ab, Cry2Ab and Cry1Fa proteins of Bacillus thuringiensis, which provides resistance to feeding damage of Lepidoptera pests (such as Ostrinia furnacalis and Spodoptera frugiperda); and it expresses the glyphosate-resistant 5-enolpyruvylshikimate-3-phosphate synthase (EPSPS) protein of Agrobacterium strain CP4, which confers plants to the glyphosate tolerance. Dual-trait maize has the following advantages: 1) protection from economic losses due to Lepidoptera pests (such as Ostrinia furnacalis, Mythimna separata, Spodoptera frugiperda, Helicoverpa armigera, Agrotis ipsilon, Conogethes punctiferalis and the likes); 2) the ability to apply glyphosate-containing agricultural herbicides to maize crops for broad-spectrum weed control; and 3) no reduction in maize yields. Specifically, the event LP026-2 of the present invention has a high resistance level to target pests, which can make the mortality rate of pests as high as 100% and protect plants to make their damage rate as low as 0%; high tolerance to glyphosate herbicide, which can protect plants and make their damage rate as low as 0%; moreover, the agronomic characters of plants containing this event are excellent, and the yield percentage can be as high as 100%. In addition, the genes encoding insect resistance and glyphosate tolerance are linked to the same DNA segment and exist at a single locus in the genome of transgenic maize event LP026-2, which improves the breeding efficiency and enables the use of molecular markers to track the transgene insertion fragments in breeding populations and their progeny. At the same time, the primer or probe sequence provided in the detection method of the present invention can generate amplification products identified as transgenic maize event LP026-2 or its progeny, and can quickly, accurately and stably identify the plant materials originating from transgenic maize event LP026-2.

### BRIEF DESCRIPTION OF THE DRAWINGS

FIG. 1 is a schematic structural diagram of the junction site between the transgene insertion sequence and the maize genome for detecting a nucleic acid sequence of a maize plant LP026-2 and a detection method thereof;

FIG. 2 is a schematic structural diagram of the recombinant expression vector pLP026 used for detecting the nucleic acid sequence of maize plant LP026-2 and the detection method thereof;

FIG. 3 shows the in vitro resistance effect of transgenic maize comprising transgenic maize event LP026-2 on Lepidoptera pests;

FIG. 4 is a photo showing the field effect of the transgenic maize comprising the transgenic maize event LP026-2 of the present invention on artificially inoculated Ostrinia furnacalis;

FIG. 5 is a photo showing the field effect of the transgenic maize comprising the transgenic maize event LP026-2 of the present invention on artificially inoculated Helicoverpa armigera;

FIG. 6 is a photo showing the field effect of the transgenic maize comprising the transgenic maize event LP026-2 of the present invention under the natural occurrence condition of Conogethes punctiferalis;

FIG. 7 is a photo showing the field effect of transgenic maize comprising the transgenic maize event LP026-2 under the natural occurrence conditions of Spodoptera exigua;

FIG. 8 is a photo showing the field effect of the transgenic maize comprising the transgenic maize event LP026-2 under the natural occurrence conditions of Spodoptera frugiperda; and

FIG. 9 is a photo showing the field effect of the transgenic maize comprising the transgenic maize event LP026-2 when glyphosate herbicide is sprayed at a concentration 4 times higher than the recommended spraying concentration.

### DETAILED DESCRIPTION OF THE EMBODIMENTS

The present invention will be described in further detail by the following examples. The features and advantages of the present invention will become clearer and more definite through these exemplary illustrations.

The specialized word “exemplary” here means “serving as an example, embodiment or illustration”. Any example described herein as “exemplary” is not necessarily to be construed as being superior or better than other examples.

In addition, the technical features involved in different embodiments of the present invention described below may be combined with each other as long as they do not conflict with each other.

The technical solutions of the present invention for detecting the nucleic acid sequence of maize plant LP026-2 and the method for detecting the same will be further explained through specific examples.

### Example 1: Cloning and Transformation

### 1.1: Vector Cloning

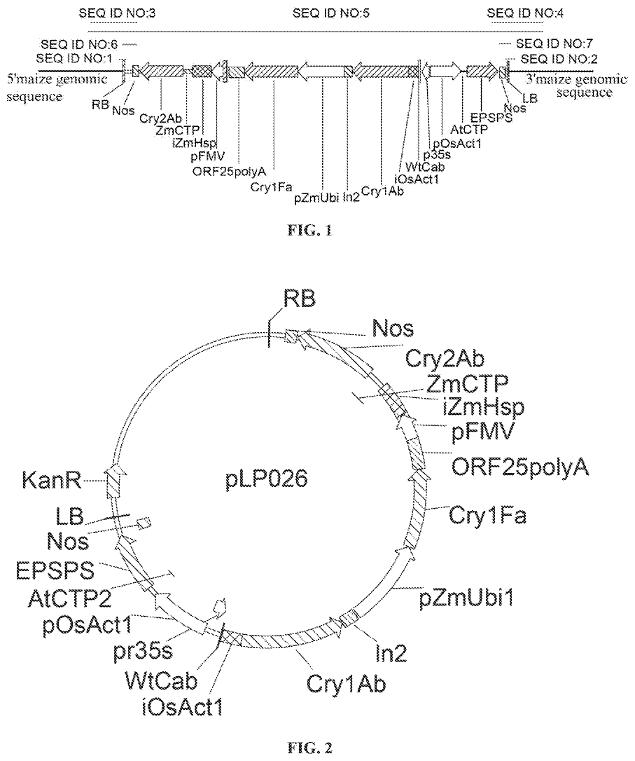

The recombinant expression vector pLP026 (as shown in FIG. 2) was constructed using standard gene cloning technology. The vector pLP026 contains four transgenic expression cassettes in series, wherein the first expression cassette is composed of the Figwort mosaic virus 35s promoter (prFMV), operatively linked to an intron of maize heat shock protein HSP70 gene (iZmHSP70), operably linked to a maize chloroplast transport peptide 2 (spZmCTP2), operably linked to an insect-resistant Cry2Ab protein of Bacillus thuringiensis (cCry2Ab), and operably linked to a transcription terminator of nopaline synthase (tNos); the second expression cassette is composed of tandem repeated promoters of maize ubiquitin gene Ubi containing an enhancer region (prZmUbi), operably linked to an insect-resistant gene Cry1Fa of Bacillus thuringiensis (cCry1Fa), and operably linked to a terminator ORF25PolyA from Agrobacterium tumefaciens pTi15955; the third expression cassette is composed of a cauliflower mosaic virus (CaMV) 35S promoter (pr35S), operably linked to the non-coding leading strand sequence at the 5′ end of the wheat chloroplast a/b binding protein (lWtCab), operably linked to the rice actin gene 1 intron (iOsAct1), operably linked to the insect-resistant Cry1Ab protein of Bacillus thuringiensis (cCry1Ab), and operably linked to the terminator of the benzenesulfonamide-inducible gene 2 (tin2); and the fourth expression cassette is composed of the rice actin 1 promoter (prOsAct1), operably linked to Arabidopsis chloroplast transporter peptide (spAtCTP2), operably linked to glyphosate-tolerant 5-enolpyruvylshikimate-3-phosphate synthase (cEPSPS) of Agrobacterium strain CP4, and operably linked to transcription terminator of nopaline synthase (tNos). The vector pLP026 was transformed into Agrobacterium LBA4404 (Invitrgen, Chicago, USA; Cat. No: 18313-015), and the transformed cells were screened using 5-enolpyruvylshikimate-3-phosphate synthase (EPSPS) as a selection marker.

### 1.2: Plant Transformation

Transformation was carried out using the conventional Agrobacterium tumefaciens infection method, wherein aseptically cultured young maize embryos were co-cultured with Agrobacterium tumefaciens as described in Example 1.1, in order to transfer the T-DNA in the constructed recombinant expression vector pLP026 into the maize genome to produce a transgenic maize event.

For Agrobacterium-mediated maize transformation, in brief, immature young embryos were separated from maize and contacted with Agrobacterium suspension, wherein Agrobacterium can transfer the nucleic acid sequences of cry1Ab, cry2Ab and cry1Fa genes and the nucleic acid sequences of epsps gene to at least one cell of one of the immature embryos (step 1: infection step), and in this step, the young embryos were specifically immersed into the Agrobacterium suspension (OD660=0.4 to 0.6, infection medium (MS salt 4.3 g/L, MS vitamin, casein 300 mg/L, sucrose 68.5 g/L, glucose 36 g/L, acetosyringone (AS) 40 mg/L, 2,4-dichlorophenoxyacetic acid (2,4-D) 1 mg/L, pH5.3) to start the inoculation. Young embryos were co-cultured with Agrobacterium for a period of time (3 days) (step 2: co-culture step). Specifically, after the infection step, young embryos were cultured in solid medium (MS salt 4.3 g/L, MS vitamin, casein 300 mg/L, sucrose 20 g/L, glucose 10 g/L, acetosyringone (AS) 100 mg/L, 2,4-dichlorophenoxyacetic acid (2,4-D) 1 mg/L and agar 8 g/L). After this co-culture stage, there may be an optional “recovery” step. In the “recovery” step, there is at least one antibiotic (e.g., cephalosporin) known to inhibit the growth of Agrobacterium in the recovery medium (MS salt 4.3 g/L, MS vitamin, casein 300 mg/L, sucrose 30 g/L, 2,4-dichlorophenoxyacetic acid (2,4-D) 1 mg/L, plant gel 3 g/L, pH 5.8), without the addition of a selective agent for the plant transformant (step 3: recovery step). Specifically, young embryos were cultured on a solid medium with antibiotics but no selective agent to eliminate Agrobacterium and provide a recovery period for infected cells. Next, the inoculated young embryos were cultured in a medium containing a selective agent (N-(phosphocarboxymethyl) glycine) and the growing transformed callus was selected (step 4: selection step). Specifically, young embryos were cultured on selecting solid medium (MS salt 4.3 g/L, MS vitamin, casein 300 mg/L, sucrose 30 g/L, N-(phosphocarboxymethyl) glycine 0.25 mol/L, 2,4-dichlorophenoxyacetic acid (2,4-D) 1 mg/L, plant gel 3 g/L, pH 5.8) with a selective agent, which resulted in selective growth of transformed cells. Then, the callus was regenerated into plants (step 5: regeneration step). Specifically, the callus grown on the medium containing the selective agent was cultured on the solid medium (MS differentiation medium and MS rooting medium) to regenerate plants.

The resistant callus obtained by selection were transferred to MS differentiation medium (MS salt 4.3 g/L, MS vitamin, casein 300 mg/L, sucrose 30 g/L, 6-benzyladenine 2 mg/L, N-(phosphocarboxymethyl) glycine 0.125 mol/L, plant gel 3 g/L, pH=5.8) and cultured for differentiation at 25° C. The differentiated seedlings were transferred to MS rooting medium (MS salt 2.15 g/L, MS vitamin, casein 300 mg/L, sucrose 30 g/L, indole-3-acetic acid 1 mg/L, agar 8 g/L, pH=5.8) and cultured at 25° C. until about 10 cm tall, and then transferred to greenhouse for cultivation until fruiting. In the greenhouse, the plants were cultured at 28° C. for 16 hours and then at 20° C. for 8 hours every day.

### 1.3: Identification and Selection of Transgenic Events

A total of 1500 independent transgenic TO individual plants were produced. By molecular detection (including detection of copy number of target gene, insertion position and the like), evaluation of target traits (insect resistance and herbicide tolerance) and agronomic traits conducted on all TO individual plants, LP026-2 was obtained after abnormal transformant plants was eliminated.

### Example 2: Detection of Transgenic Maize Event LP026-2 with TaqMan

About 100 mg leaves of transgenic maize event LP026-2 were taken as samples, and their genomic DNA was extracted with DNeasy Plant Maxi Kit of Qiagen, and the copy numbers of cry1Ab, cry2Ab, cry1Fa and epsps were detected by Taqman probe fluorescence quantitative PCR method. At the same time, wild maize plants were used as control, and the detection and analysis were carried out according to the above methods. The experiment was set up with three replications and the average value was taken.

The specific method is as follows:
Step 11, 100 mg leaves of transgenic maize event LP026-2 were taken, ground into homogenate in a mortar with liquid nitrogen, and 3 replicates were taken for each sample;
Step 12, genomic DNA of the above samples was extracted by using DNeasy Plant Mini Kit of Qiagen, and for specific methods, product manual may be referred to;
Step 13, the genomic DNA concentration of the above samples was determined with NanoDrop 2000 (Thermo Scientific);
Step 14, the genomic DNA concentration of the above samples was adjusted to the same concentration value, wherein the concentration value ranges from 80 to 100 ng/μl;
Step 15, the copy number of the samples was identified by Taqman probe fluorescence quantitative PCR method, the identified sample with a known copy number was taken as the standard and the sample of wild maize plant as the control, wherein each sample has 3 replicates, and the average value thereof were taken; the sequences of the primers and probes for fluorescence quantitative PCR were as follows respectively:

The following primers and probe were used to detect the cry1Ab gene sequence:

Primer 1:
TGGGAGGACGGAATGATATTG, as shown in the sequence
listing under SEQ ID NO: 16;
Primer 2:
AACTCGTCCGTGAGCATCATC, as shown in the sequence
listing under SEQ ID NO: 17;
Probe 1:
AACTCCGCGCTGCGATGAATCC, as shown in the sequence
listing under SEQ ID NO: 18;

The following primers and probe were used to detect the cry2Ab gene sequence:

Primer 3:
GGACAGAGGCACCGCATT, as shown in the sequence
listing under SEQ ID NO: 19;
Primer 4:
CGGGTCTGCAAGCAAACG, as shown in the sequence
listing under SEQ ID NO: 20;
Probe 2:
TCCACTTGGCGGTTGAACTCCTCC, as shown in the sequence
listing under SEQ ID NO: 21;

The following primers and probe were used to detect the cry1 Fa gene sequence:

Primer 5:
GCTATGTCCAGTCCCCAACCT, as shown in the sequence
listing under SEQ ID NO: 22;
Primer 6:
CAAGCTGCTAACCTGCACTTGT, as shown in the sequence
listing under SEQ ID NO: 23;
Probe 3:
CCCAAACGACACAGCGTCGCG, as shown in the sequence
listing under SEQ ID NO: 24;

The following primers and probe were used to detect the epsps gene sequence:

Primer 7:
GCAAATCCTCTGGCCTTTCC, as shown in the sequence
listing under SEQ ID NO: 25;
Primer 8:
TGAAGGACCGGTGGGAGAT, as shown in the sequence
listing under SEQ ID NO: 26;
Probe 4:
CGTCCGCATTCCCGGCGA, as shown in the sequence
listing under SEQ ID NO: 27;

The PCR reaction system was as follows:

| | | |
|---|---|---|
| JumpStart ™ Taq ReadyMix ™ (Sigma) | 10 | μL |
| 50 × primer/probe Mixture | 1 | μL |
| genomic DNA | 3 | μL |
| water (ddH₂O) | 6 | μL |

The 50×primer/probe Mixture contained 45 μL of each primer with a concentration of 1 mM, 50 μL of probes with a concentration of 100 μM and 860 μL of 1×TE buffer, and was stored in an amber test tube at 4° C.

The PCR reaction conditions were as follows:

| step | temperature | time | |
|---|---|---|---|
| 21 | 95° C. | 5 | minutes |
| 22 | 95° C. | 30 | seconds |
| 23 | 60° C. | 1 | minute |
| 24 | back to step 22 and repeat | | |
| | for 40 times | | |

Data were analyzed using SDS2.3 software (Applied Biosystems) to obtain a single-copy transgenic maize event LP026-2.

### Example 3: Detection of Transgenic Maize Event LP026-2

### 3.1: Extraction of the Genomic DNA

The DNA was extracted according to the conventional CTAB (cetyltrimethyl ammonium bromide) method: 2 grams of leaves of young transgenic maize event LP026-2 were ground into powder in liquid nitrogen, and then 0.5 mL of preheated CTAB Buffer [20 g/L CTAB, 1.4M NaCl, 100 mM Tris-HCl, 20 mM EDTA (ethylenediamine tetraacetic acid)] at 65° C. was added to extract DNA, and the pH was adjusted with NaOH to 8.0, the resultant was mixed thoroughly, and then extracted at a temperature of 65° C. for 90 min; 0.5 times volume of phenol and 0.5 times volume of chloroform were added, and mixed evenly by inversion; the resultant was subjected to centrifugation at 12,000 rpm for 10 min; the supernatant was absorbed, 1 volume of isopropanol was added, and the centrifuge tube was gently shaken, and stood at −20° C. for 30 min; the resultant was subjected to centrifugation for 10 min at 12,000 rpm; DNA was collected at the bottom of the tube; the supernatant was discarded, and the precipitate was washed with 0.5 mL of 70% ethanol by volume; the resultant was subjected to centrifugation at 12,000 rpm for 5 minutes; and vacuum pumped or blow-dried on ultra-clean table; DNA precipitate was dissolved in a proper amount of TE buffer (10 mM Tris-HCl, 1 mM EDTA, pH 8.0) and stored at −20° C.

### 3.2: Analysis on the Flanking DNA Sequence

The concentration of the extracted DNA sample was determined, and the concentration of the sample to be detected was made to be between 80 ng/μL and 100 ng/μL. Genomic DNA was digested with the selected restriction enzymes SpeI, PstI, BssHII (5′ end analysis) and SacI, KpnI, XmaI and NheI (3′ end analysis). 26.5 μL of genomic DNA, 0.5 μL of the restriction endonuclease selected above and 3 μL of enzyme digestion buffer were added to each enzyme digestion system, and the enzyme digestion was carried out at a proper temperature for 1 hour. After the enzyme digestion was finished, 70 μL of anhydrous ethanol was added into the enzyme digestion system, ice bath was performed for 30 min, centrifugation was performed at 12,000 rpm for 7 min, the supernatant was discarded and the participate was blown dried, followed by the addition of 8.5 μL of double-distilled water (ddH2O), 1 μL of 10×T4 buffer and 0.5 μL of T4 ligase to ligate overnight at 4° C. 5′ and 3′ transgenic/genomic DNA were separated by PCR amplification with a series of nested primers. Specifically, the primer set for isolating 5′ transgenic/genomic DNA includes SEQ ID NO: 13 and SEQ ID NO:34 as the first primers, SEQ ID NO:35 and SEQ ID NO:36 as the second primers, and SEQ ID NO: 13 as the sequencing primer. The primer set for isolating 3′ transgenic/genomic DNA includes SEQ ID NO:15 and SEQ ID NO:37 as the first primers, SEQ ID NO:38 and SEQ ID NO:39 as the second primers, and SEQ ID NO:15 as the sequencing primer. The PCR reaction conditions are shown in Table 3.

The obtained amplicons were electrophoresed on 2.0% agarose gel to separate PCR reactants, and then the target fragment was separated from the agarose substrate by using the QIAquick Gel extraction kit (Catalog #_28704, Qiagen Inc, Valencia, CA). The purified PCR products were then sequenced (e.g., by ABI Prism™ 377, PE Biosystems, Foster City, CA) and analyzed (e.g., by Sequence Analysis Software of DNASTAR, DNASTAR Inc., Madison, WI).

The 5′ and 3′ flanking sequences and junction sequences were confirmed by standard PCR method. The 5′ flanking sequence and the junction sequence may be confirmed by using SEQ ID NO:8 or SEQ ID NO: 12, in combination with SEQ ID NO:9, SEQ ID NO:13 or SEQ ID NO: 34. The 3′ flanking sequence and the junction sequence may be confirmed by using SEQ ID NO:11 or SEQ ID NO:14, in combination with SEQ ID NO:10, SEQ ID NO:15 or SEQ ID NO:37. The PCR reaction system and amplification conditions are shown in Table 3. A person skilled in the art will understand that other primer sequences can also be used to confirm flanking sequences and junction sequences.

The sequencing for the DNA of PCR products provides DNA that may be used to design other DNA molecules, wherein said other DNA molecules are used as primers and probes for the identification of maize plants or seeds derived from transgenic maize event LP026-2.

It was found that nucleotides 1-995 of SEQ ID NO:5 showed the maize genomic sequence flanking the right border of the insertion sequence of transgenic maize event LP026-2 (5′ flanking sequence), and the nucleotides 17152-17487 of SEQ ID NO:5 showed the maize genomic sequence flanking the left border of the insertion sequence of transgenic maize event LP026-2 (3′ flanking sequence). The sequence of 5′ junction site is listed in SEQ ID NO: 1, and the sequence of 3′ junction site is listed in SEQ ID NO:2.

### 3.3: PCR Assay on Junction

The junction sequence was a relatively short polynucleotide molecule, which is a new DNA sequence and is diagnostic for the DNA of the transgenic maize event LP026-2 when detected in the nucleic acid detection analysis. The junction sequence of SEQ ID NO:1 was composed of 11 bp on each side of the insertion site of transgenic maize event LP026-2, 11 bp on the T-DNA RB region side and 11 bp on the maize genomic DNA side, and the junction sequence of SEQ ID NO:2 was composed of 11 bp on each side of the insertion site of transgenic maize event LP026-2, 11 bp on the T-DNA LB region side and 11 bp on the maize genomic DNA side. Longer or shorter polynucleotide junction sequences may be selected from SEQ ID NO:3 or SEQ ID NO:4. Junction sequences (SEQ ID NO:1 in the 5′ junction region, and SEQ ID NO:2 in the 3′ junction region) were useful as DNA probes or as DNA primer molecules in DNA detection methods. The junction sequences SEQ ID NO:6 and SEQ ID NO:7 were also new DNA sequences in transgenic maize event LP026-2, which may also be used as DNA probes or DNA primer molecules to detect the DNA in transgenic maize event LP026-2. The SEQ ID NO: 6 (nucleotides 996-1495 of SEQ ID NO: 3) spans the DNA sequence of the LP026 construct and the tNos transcription termination sequence, and the SEQ ID NO: 7 (nucleotides 1-254 of SEQ ID NO: 4) spans the tNos transcription termination sequence and the LP026 construct DNA sequence.

In addition, amplicons were generated by using at least one primer from SEQ ID NO: 3 or SEQ ID NO:4, which, when used in a PCR method, diagnostic amplicons for transgenic maize event LP026-2 can be generated.

Specifically, a PCR product was generated from the 5′ end of the transgene insertion sequence, which comprises a portion of the genomic DNA flanking the 5′ end of the T-DNA insertion sequence in the genome of plant materials derived from the transgenic maize event LP026-2. This PCR product contains SEQ ID NO:3. To perform PCR amplification, a primer 11 (SEQ ID NO:8) which hybridizes with the genomic DNA sequence flanking the 5′ end of the transgene insertion sequence and a primer 12 (SEQ ID NO:9) which is paired with it and located in the transcription termination sequence of the transgene tNos were designed.

A PCR product was generated from the 3′ end of the transgene insertion sequence, which contains a portion of genomic DNA flanking the 3′ end of the T-DNA insertion sequence in the genome of the plant material derived from the transgenic maize event LP026-2. This PCR product contains SEQ ID NO:4. To perform PCR amplification, a primer 14 (SEQ ID NO:11) which hybridizes with the genomic DNA sequence flanking the 3′ end of the transgene insertion sequence and a primer 13 (SEQ ID NO:10) which is paired with the tNos transcription termination sequence at the 3′ end of the insertion sequence were designed.

The DNA amplification conditions illustrated in Tables 3 and 4 may be used in the above-mentioned PCR assay on junction to generate a diagnostic amplicon for transgenic maize event LP026-2. Amplicons may be detected by using Stratagene Robocycle, MJ Engine, Perkin-Elmer 9700 or Eppendorf Mastercycler Gradien thermal cycler, or by methods and equipment known to a person skilled in the art.

TABLE 3
Conditions for identification of 5′ transgene inserts/genome junction
regions of transgenic maize event LP026-2 on PCR steps and reaction mixture

| Step | Reagent | Quantity | Note |
|---|---|---|---|
| 1 | Nuclease-free water | Added to a final | |
| | | volume of 20 μL | |
| 2 | 10*reaction buffer(with MgCl₂) | 2.0 μL | 1* final concentration of |
| | | | buffer with a final |
| | | | concentration of 1.5 mM |
| | | | MgCl₂ |
| 3 | 10 mM solution of dATP, dCTP, dGTP and | 0.4 μL | Final concentartion of |
| | dTTP | | 200 μM for each dNTP |
| 4 | Event Primer 11 (SEQ ID NO: 8 suspended | 0.2 μL | A final concentration of |
| | in 1*TE buffer or nuclease-free water to a | | 0.1 μM |
| | concentration of 10 μM) | | |
| 5 | Event Primer 12 (SEQ ID NO: 9 suspended | 0.2 μL | A final concentration of |
| | in 1*TE buffer or nuclease-free water to a | | 0.1 μM |
| | concentration of 10 μM) | | |
| 6 | Rnase, no DNase(500 ng/mL) | 0.1 μL | 50 ng/reaction |
| 7 | Extracted DNA(template): leaves of the | 1.0 μL(it is | 1 unit/reaction |
| | sample to be analyzed | recommended to | |
| | | change new pipette | |
| | | before next step) | |
| 8 | Extracted DNA(template): leaves of the | 200 ng of genomic | |
| | sample to be analyzed | DNA | |
| | Negative control | 50 ng of non-GM | |
| | | maize genomic DNA | |
| | Negative control | No template | |
| | | DNA(solution in | |
| | | which DNA is | |
| | | resuspended) | |
| | positive control | 50 ng maize genomic | |
| | | DNA containing | |
| | | LP026-2 | |

TABLE 4
Conditions on Perkin-Elmer 9700 Thermal Cycler

| Number | |
|---|---|
| of cycle | Setting |
| 1 | 94° C. for 3 minutes |
| 34 | 94° C. for 30 seconds |
| | 64° C. for 30 seconds |
| | 72° C. for 1 minutes |
| 1 | 72° C. for 10 minutes |

Gently mixing was performed and 1 to 2 drops of mineral oil may be added on top of each reaction solution if there is no holding cap on the thermal cycler. PCR was performed using the parameters for each cycle shown in Table 4 on Stratagene Robocoller (Stratagene, La Jolla, California), MJ Engine (MJ R-Biorad, Hercules, California), Perkin-Elmer 9700 (Perkin Elmer, Boston, MA) or Eppendorf Mastercycler Gradient (Eppendorf, Hamburg, Germany). MJ Engine or Eppendorf Mastercycler Gradient thermal cycler should be run in calculation mode. Perkin-Elmer 9700 thermal cycler should be run with the ramp speed set to the maximum value.

The experimental results showed that primers 11 and 12 (SEQ ID NOs: 8 and 9), when used in the PCR reaction of genomic DNA of transgenic maize event LP026-2, yielded an amplification product of 1495 bp fragment, but when used in the PCR reaction of untransformed maize genomic DNA and non-LP026-2 maize genomic DNA, no fragment was amplified; and primers 13 and 14 (SEQ ID NOs: 10 and 11), when used in the PCR reaction of genomic DNA of transgenic maize event LP026-2, yielded an amplification product of 590 bp fragment, and when used in the PCR reaction of untransformed maize genomic DNA and non-LP026-2 maize genomic DNA, no fragment was amplified.

PCR assay on junction may also be used to identify whether the material from transgenic maize event LP026-2 is homozygous or heterozygous. Primer 15 (SEQ ID NO:12), primer 16 (SEQ ID NO:13) and primer 17 (SEQ ID NO:14) or primer 16 (SEQ ID NO:13), primer 17 (SEQ ID NO:14) and primer 18 (SEQ ID NO:15) were used for amplification reaction to generate diagnostic amplicons for transgenic maize event LP026-2. The DNA amplification conditions illustrated in Tables 5 and 6 may be used in the above-mentioned junction assay to generate a diagnostic amplicon of transgenic maize event LP026-2.

TABLE 5
Reaction solutions for junction assay

| Step | Reagent | Quantity | Note |
|---|---|---|---|
| 1 | Nuclease-free water | Added to a final | |
| | | volume of 5 μL | |
| 2 | 2*Universal Master Mix(Applied Biosystems Catalog | 5 μL | 1* final |
| | No. 43044374304437) | | concentration |
| 3 | Primers 15 (SEQ ID NO: 12), 16(SEQ ID NO: 13) and | 0.3 μL | A final |
| | 17 (SEQ ID NO: 14) (resuspended in nuclease-free | | concentration |
| | water to a concentration of 10 μM) | | of 0.1 μM |
| 4 | REDTaq DNA Polymerase (1 unit/μL) | 1.0 μL(it is | 1 unit/reaction |
| | | recommended to | |
| | | change new pipette | |
| | | before next step) | |
| 5 | Extracted DNA (template): leaves of samples to be | 200 ng genomic | |
| | analyzed | DNA | |
| | Negative control | 50 ng of non-GM | |
| | | maize genomic | |
| | | DNA | |
| | Negative control | No template | |
| | | DNA(solution in | |
| | | which DNA is | |
| | | resuspended) | |
| | Positive control | 50 ng maize genomic | |
| | | DNA containing | |
| | | LP026-2 | |

TABLE 6
Conditions for Perkin-Elmer 9700 thermal cycler for junction assay

| Number of cycle | Setting |
|---|---|
| 1 | 95° C. for 10 minutes |
| 10 | 95° C. for 15 seconds |
| | 64° C. for 1 minute(−1° C./cycle) |
| 25 | 95° C. for 15 seconds |
| | 54° C. for 1 minute |
| 1 | Immersion at 10° C. |

PCR was performed with the parameters for each cycle shown in Table 6 on Stratagene Robocycler (Stratagene, La Jolla, CA), MJ Engine (MJ R-Biorad, Hercules, CA), Perkin-Elmer 9700 (Perkin Elmer, Boston, MA) or Eppendorf Mastercycler Gradient (Eppendorf, Hamburg, Germany). MJ Engine or Eppendorf Mastercycler Gradient thermal cycler should be run in calculation mode. Perkin-Elmer 9700 thermal cycler should be run with the ramp speed set to the maximum value.

In the amplification reaction, the biological sample containing template DNA contains DNA that can be used for diagnosing the transgenic maize event LP026-2 in the sample. Or the reaction will generate two different DNA amplicons from the biological sample containing DNA from maize genome, wherein the DNA from the maize genome is heterozygous with respect to the allele corresponding to the inserted DNA existing in the transgenic maize event LP026-2. These two different amplicons will correspond to a first amplicon from the wild-type maize genome locus and a second amplicon for diagnosing the transgenic maize event LP026-2 DNA. Only a maize DNA sample generate a single amplicon corresponding to the second amplicon described for the heterozygous genome is diagnostically determinable for the presence of the transgenic maize event LP026-2 in the sample, and the sample is generated by maize seeds which are homozygous with respect to the allele corresponding to the inserted DNA existing in the transgenic maize plant LP026-2.

It should be noted that the pair of primers for the transgenic maize event LP026-2 was used to generate an amplicon which was diagnostic for the genomic DNA of the transgenic maize event LP026-2. These pairs of primers include, but are not limited to, primers 11 and 12 (SEQ ID NOs: 8 and 9) and primers 13 and 14 (SEQ ID NOs: 10 and 11) used in the DNA amplification method. In addition, control primers 9 and 10 (SEQ ID NO:28 and SEQ ID NO:29) for amplifying maize endogenous genes were included as an intrinsic standard of reaction conditions. The analysis of the samples of DNA extract of transgenic maize event LP026-2 should include a positive tissue DNA extract control of transgenic maize event LP026-2, a negative DNA extract control from non-transgenic maize event LP026-2 DNA and a negative control without template maize DNA extract. In addition to these pairs of primers, any pair of primers from SEQ ID NO:3 or SEQ ID NO:4, or their complementary sequences, may be used, which, when used in DNA amplification reaction to generate amplicons containing SEQ ID NO:1 or SEQ ID NO:2, respectively, that are diagnostic for tissues derived from transgenic maize plant LP026-2. The DNA amplification conditions illustrated in Tables 2 to 5 may be used to generate diagnostic amplicons of transgenic maize event LP026-2 using appropriate pair of primers. Extracts of maize plants or seeds presumed to contain DNA of maize plant or seed comprising the transgenic maize event LP026-2, which generate amplicons diagnostic for the transgenic maize event LP026-2 when tested in a DNA amplification method, or products derived from the transgenic maize event LP026-2, may be used as templates for amplification to determine whether the transgenic maize event LP026-2 is present.

### Example 4: Detection of Transgenic Maize Event LP026-2 by Southern Blot Hybridization

### 4.1. DNA Extraction for Southern Blot Hybridization

Southern blot analysis was carried out by using homozygous transformation events from the T4 and T5 generations. About 5 to 10 g of plant tissue was ground in liquid nitrogen using a mortar and pestle. Plant tissues were resuspended in 12.5 mL of extraction buffer A (0.2M Tris pH=8.0, 50 mM EDTA, 0.25M NaCl, 0.1% v/v β-mercaptoethanol, 2.5% w/v polyvinyl pyrrolidone) and centrifuged at 4,000 rpm for 10 minutes (2755 g). After discarding the supernatant, the precipitate was resuspended in 2.5 mL of extraction buffer B (0.2M Tris pH=8.0, 50 mM EDTA, 0.5M NaCl, 1% v/v β-mercaptoethanol, 2.5% w/v polyvinyl pyrrolidone, 3% N-Lauroylsarkosin and 20% ethanol) and incubated at 37° C. for 30 minutes. During the incubation period, the samples were mixed once with sterile rings. After incubation, equal volume of chloroform/isoamyl alcohol (24:1) was added, gently mixed by inversion, and centrifuged at 4,000 rpm for 20 minutes. The aqueous layer was collected and centrifuged at 4,000 rpm for 5 minutes after adding 0.54×volume of isopropanol to precipitate DNA. The supernatant was discarded and the DNA precipitate was resuspended in 500 μL TE. In order to degrade any RNA present, DNA and 1 μL of 30 mg/ml RNAaseA were incubated at 37° C. for 30 minutes, centrifuged at 4,000 rpm for 5 minutes, and DNA was precipitated by centrifugation at 14,000 rpm for 10 minutes in the presence of 0.5×volume of 7.5M ammonium acetate and 0.54×volume of isopropanol. After discarding the supernatant, the precipitate was washed with 500 μL of 70% ethanol, dried and resuspended in 100 μL of TE.

### 4.2. Digestion by Restriction Enzyme

DNA concentration was quantified using a spectrophotometer or fluorometer (using 1×TAE and GelRED dyes). In 100 μL reaction system, 5 μg DNA was digested each time. Genomic DNA was digested with restriction endonucleases BamHI and HindIII respectively, and partial sequences of Cry2Ab and EPSPS on T-DNA were used as probes; genomic DNA was digested with restriction enzymes AvrII and HindIII, respectively, and partial sequences of Cry1Ab and Cry1Fa on T-DNA were used as probes. For each enzyme, the digests were incubated overnight at an appropriate temperature. The sample was rotated by a vacuum centrifugal evaporation concentrator (speed vacuum) to reduce the volume to 30 μL.

### 4.3. Gel Electrophoresis

Bromophenol blue dye was added to each sample from the Example 4.2, and each sample was loaded onto a 0.7% agarose gel containing ethidium bromide, separated by electrophoresis in TBE electrophoresis buffer, and the gel was electrophoresed overnight at 20 volts.

The gel was washed in 0.25M HCl for 15 minutes to depurinate the DNA, and then washed with water. Southern blot hybridization was set as follows: 20 sheets of dry thick blotting paper were placed in a tray, and 4 sheets of dry thin blotting paper were placed on it. One sheet of thin blotting paper was pre-moistened in 0.4M NaOH, and placed on the paper pile, followed by one sheet of Hybond-N+ transfer membrane (Amersham pharmacia Biotechnology, #RPN303B) pre-moistened in 0.4M NaOH. The gel was placed on top of the membrane and it was ensured that there were no air bubbles between the gel and the membrane. 3 additional pre-soaked blotting papers were placed on top of the gel and the buffer tray was filled with 0.4M NaOH. The gel stack and the buffer tray were connected with a wick pre-soaked in 0.4M NaOH, and DNA was transferred to the membrane. DNA transfer was carried out at room temperature for about 4 hours. After transfer, the Hybond membrane was rinsed in 2×SSC for 10 seconds, and DNA was bound to the membrane through UV crosslinking.

### 4.4. Hybridization

The suitable DNA sequences were amplified by PCR for probe preparation. The DNA probes were SEQ ID NO:30, SEQ ID NO:31, SEQ ID NO:32 and SEQ ID NO:33, or were partially homologous or complementary to the above sequences. 25 ng of probe DNA was boiled in 45 μL TE for 5 minutes, placed on ice for 7 minutes, and then transferred to RediprimeII (Amersham Pharmacia Biotech, #RPN1633) test tube. After addition of 5 μl of 32P-labeled dCTP to Rediprime tube, the probe was incubated at 37° C. for 15 minutes. According to the manufacturer's instructions, the probe was purified by centrifugation through a microcentrifugation G-50 column (Amersham Pharmacia Biotech, #27-5330-01) to remove unincorporated dNTPs. The probe activity was measured by scintillation counter. The Hybond membrane was pre-hybridized by wetting it with 20 mL of pre-warmed Church pre-hybridization solution (500 mM Na3PO4, 1 mM EDTA, 7% SDS and 1% BSA) at 65° C. for 30 minutes. The labeled probes were boiled for 5 minutes and placed on ice for 10 minutes. An appropriate amount of probe was added to the prehybridization buffer (1 million counts per mL of pre-hybridization buffer), and hybridization was performed at 65° C. overnight. The next day, the hybridization buffer was discarded, the membrane was washed with 20 ml of Church washing solution 1 (40 mM Na3PO4, 1 mM EDTA, 5% SDS and 0.5% BSA), and then the membrane was washed in 150 mL of Church washing solution 1 for 20 minutes at 65° C. The process was repeated twice with Church washing solution 2 (40 mM Na3PO4, 1 mM EDTA and 1% SDS). The membrane was exposed to a phosphor screen or X-ray film to detect the location where the probe bound.

Two control samples were included in each Southern assay: (1) DNA from negative (untransformed) segregant, which was used to identify any endogenous maize sequence that could hybridize with element-specific probes; (2) DNA from positive segregant, in which pLP026 digested by HindIII was introduced, and its amount was equivalent to a single copy based on the probe length, so as to illustrate the sensitivity of this experiment in detecting a single-copy gene in maize genome.

The hybridization data provided conclusive evidence to support TaqMan™ PCR analysis, that is, maize plant LP026-2 contained a single copy of Cry2Ab, Cry1Fa, Cry1Ab and EPSPS genes. Using the Cry2Ab probe, digestion by BamHI and HindIII produced individual bands with a size of about 4.4 kb and 4.9 kb respectively; using Cry1Fa probe, digestion by AvrII and HindIII produced individual bands with a size of about 9.4 kb and 17.3 kb, respectively; using Cry1 Ab probe, digestion by AvrII and HindIII produced individual bands with a size of about 9.0 kb and 17.3 kb, respectively; and using the EPSPS probe, digestion by BamHI and HindIII produced single bands with a size of about 4.0 kb and 17.3 kb, respectively. This indicates that one copy each of Cry1Ab, Cry2Ab, Cry1Fa and EPSPS is present in the maize transformation event LP026-2.

### Example 5: Detection on Insect Resistance

### 5.1. Bioassay of Maize Plant LP026-2

Two plants, transgenic maize event LP026-2 and wild-type maize plant (non-transgenic, transformation recipient control (CK-)) were bioassayed for resistance to Ostrinia furnacalis, Spodoptera frugiperda, Conogethes punctiferalis, Athetis lepigone, Mythimna separata, Agrotis ipsilon, Helicoverpa armigera and Spodoptera exigua according to the following method:

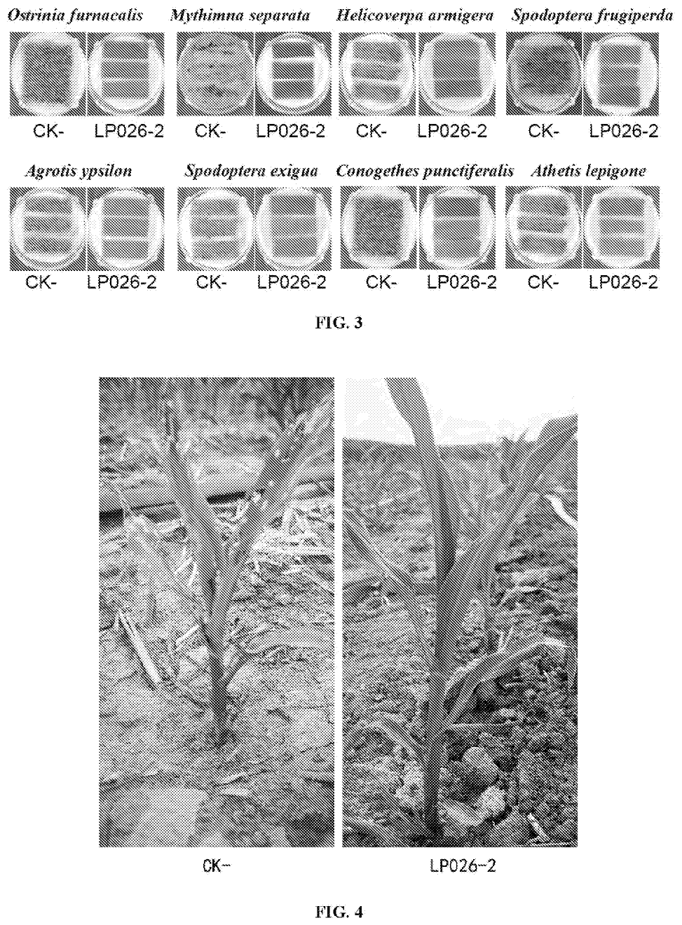

Fresh leaves (V3-V4 period) from two kinds of plants, transgenic maize event LP026-2 and wild-type maize plant (non-transgenic, transformation recipient control (CK-)), were taken respectively, rinsed well with sterile water and sucked dry with absorbent paper, and then the maize leaves were removed from the leaf veins while cutting into long strips of about 1 cm×3 cm in size, and 1 to 3 leaves (the number of leaves is determined according to the insect's food consumption) of the long strips after cutting were put a the filter paper at the bottom of a circular plastic Petri dish. The filter paper was wetted with distilled water, and 10 artificially reared larvae that are newly hatched were inoculated into each Petri dish. The insect test dish was covered, and then placed under the conditions of temperature of 26-28° C., relative humidity of 70% to 80% and photoperiod of 16:8 (light/dark) for 5 days, then the statistical result was made. The statistical mortality rate (mortality rate=(number of dead insects/number of tested insects)×100%) was calculated to characterize the level of resistance, and the results were shown in Table 7 and FIG. 3.

TABLE 7
Results of ex-vitro insect resistance bioassay for
transgenic maize event LP026-2: Mortality (%)

| insects/plants | LP026-2 | CK- |
|---|---|---|
| Ostrinia furnacalis | 100 | 2 |
| Helicoverpa armigera | 100 | 1 |
| Spodoptera frugiperda | 100 | 2 |
| Mythimna separata | 100 | 1 |
| Spodoptera exigua | 100 | 2 |
| Agrotis ypsilon | 100 | 1 |
| Conogethes punctiferalis | 100 | 1 |
| Athetis lepigone | 100 | 2 |

### 5.2. Determination of Insect-Resistant Effect of Transgenic Maize Event LP026-2 in the Field.

(1) Ostrinia furnacalis

The resistance of transgenic maize event LP026-2 to Ostrinia furnacalis, the main target pest in the field, was identified using a method of insect inoculation to living plants. Insects were inoculated at the 4-6 leaf stage and the silking stage (3-5 cm for female ear) of maize, twice in each stage with an interval of one week between the two inoculations and 50 insects inoculated each time. 14 days after inoculation at the whorl stage, the feeding situation of the middle and upper leaves of maize plants by Ostrinia furnacalis was investigated one by one, and the leaf feeding level by Ostrinia furnacalis was recorded. After inoculation at the silking stage, the damage degree of female ear and plant was investigated before harvest, including the damage length of female ear, the number of wormholes, the length of wormhole tunnel, the age and the number of surviving larvae. The resistance of transgenic maize event LP026-2 to Ostrinia furnacalis was evaluated with the index of “grading standard of Ostrinia furnacalis's damage degree to maize whorl”, and the results are shown in FIG. 4 and Table 12. During the silking stage, the cob dissection was investigated, and the statistical results are shown in Table 13. The results showed that the average leaf-feeding grade of transgenic maize event LP026-2 was significantly lower than that of transformation recipient control (CK-) at the whorl stage; and at the silking stage, the female ear damage rate, the number of larvae surviving, the tunnel length and the female ear damage level of transgenic maize event LP026-2 were significantly lower than those of transformation recipient control (CK-). Overall, the transgenic maize event LP026-2 has good resistance to Ostrinia furnacalis both at the whorl stage and silking stage.

TABLE 8
Grading criteria of Ostrinia furnacalis's
damage degree to maize whorl

| leaf-feeding grade | Description on the symptoms |
|---|---|
| 1 | The leaves are not damaged, or only the |
| | leaves have needle-like (≤1 mm) wormholes |
| 2 | a few bullet-sized wormholes (≤5 mm) |
| | on only a few leaves |
| 3 | Minority leaves have bullet-sized wormholes (≤5 mm) |
| 4 | Notches on only a few leaves (≤10 mm) |
| 5 | Notches on minority leaves (≤10 mm) |
| 6 | Notches on partial leaves (≤10 mm) |
| 7 | Only a few leaves are partially eaten, and |
| | minority leaves have large notches (≤10 mm) |
| 8 | Minority leaves are eaten, and partial |
| | leaves have large notches (≤10 mm) |
| 9 | Most leaves are eaten |

TABLE 9
Evaluation criteria of maize resistance to Ostrinia furnacalis

| average leaf-feeding grade at | |
|---|---|
| whorl stage | Resiatance level |
| 1.0-2.9 | Highly resistant (HR) |
| 3.1-4.9 | Resistant(R) |
| 5.0-6.9 | Medium resistant(MR) |
| 7.0-8.9 | Susceptible (S) |
| 9.0 | Highly susceptible (HS) |

TABLE 10
Grading criteria for damage degree of maize by Ostrinia furnacalis at ear stage

| Damage level | |
|---|---|
| of female ear | Description on the symptoms |
| 1 | No damage on female ear |
| 2 | Filaments were dameged by <50% |
| 3 | Most of filaments were dameged by ≥50%; there are larvae survived, |
| | age ≤2 instars |
| 4 | Damaged on tip of the ear by ≤1 cm; there are larvae survived, age ≤3 |
| | instars |
| 5 | Damaged on tip of the ear by ≤2 cm; or there are larvae survived, age ≤4 |
| | instars; tunnel length ≤2 cm |
| 6 | Damaged on tip of the ear by ≤3 cm; or there are larvae survived, age >4 |
| | instars; tunnel length ≤4 cm |
| 7 | Damaged on tip of the ear by ≤4 cm; tunnel length ≤6 cm |
| 8 | Damaged on tip of the ear by ≤5 cm; tunnel length ≤8 cm |
| 9 | Damaged on tip of the ear by >5 cm; tunnel length >8 cm |

TABLE 11
Evaluation criteria for resistance of maize
to Ostrinia furnacalis at ear stage

| Average damage level of female ear | Resistance type |
|---|---|
| 1-2.0 | Highly resistant (HR) |
| 2.1-3.0 | Resistant(R) |
| 3.1-5.0 | Medium resistant (MR) |
| 5.1-7.0 | Susceptible (S) |
| ≥7.1 | Highly susceptible (HS) |

TABLE 12
Resistance results of transgenic maize event LP026-2
to Ostrinia furnacalis at whorl stage

| Item/plant | LP026-2 | CK- |
|---|---|---|
| Average leaf-feeding grade | 1.06 | 8.2 |
| Resistance level | Highly resistant | Highly susceptible |

TABLE 13
Resistance of transgenic maize event LP026-2
to Ostrinia furnacalis at silking stage

| Item/plant | LP026-2 | CK- |
|---|---|---|
| Damage rate of female ear (%) | 0 | 100 |
| Larva survival number | 0 | 14 |
| Tunnel length (cm) | 0 | 3.3 |
| Damage level of female ear | 0 | 6.5 |
| Resistance level | Highly resistant | Susceptible |

(2) Mythimna separata

Artificial inoculation was carried out at the whorl stage (4-6 leaf stage) of maize for a total of 2 times, and 20 heads of artificially reared second-instar Mythimna separata were inoculated in whorl of each maize. 3 days after inoculation, the second inoculation was carried out, with the same number as the first inoculation. 14 days after inoculation, the damage degree of maize leaves by Mythimna separata was investigated. According to the damage degree of maize leaves by Mythimna separata, the average damage level (leaf-eating level) of Mythimna separata to maize leaves in each plot was calculated, and the judgment criteria was shown in Table 14, and then the resistance level of maize to Mythimna separata was judged according to the criteria in Table 15. The results of resistance to Mythimna separata at the whorl stage of transgenic maize event LP026-2 were shown in Table 16. The results showed that the transgenic maize event LP026-2 had a good level of resistance to Mythimna separata, and the notch ratio and leaf-feeding level of transgenic maize event LP026-2 were significantly lower than those of transformation recipient control (CK-).

TABLE 14
Grading criteria of damage on maize leaves by Mythimna separata

| Damage level of female ear | Description on the symptoms |
|---|---|
| 1 | The leaves are not damaged, or only the leaves have needle-like |
| | (≤1 mm) wormholes |
| 2 | a few bullet-sized wormholes (≤5 mm) on only a few leaves |
| 3 | Minority leaves have bullet-sized wormholes (≤5 mm) |
| 4 | Notches on only a few leaves (≤10 mm) |
| 5 | Notches on minority leaves (≤10 mm) |
| 6 | Notches on partial leaves (≤10 mm) |
| 7 | Only a few leaves are partially eaten, and minority leaves have |
| | large notches (≤10 mm) |
| 8 | Minority leaves are eaten, and partial leaves have large notches |
| | (≤10 mm) |
| 9 | Most leaves are eaten |

TABLE 15
Evaluation criteria of maize resistance to Mythimna separata

| Average damage level of female ear | Resistance type |
|---|---|
| 1.0-2.0 | Highly resistant (HR) |
| 2.1-4.0 | Resistant (R) |
| 4.1-6.0 | Medium resistant (MR) |
| 6.1-8.0 | Susceptible (S) |
| 8.1-9.0 | Highly susceptible (HS) |

TABLE 16
Resistance results of transgenic maize event LP026-2
to Mythimna separata at whorl stage

| Item/plant | LP026-2 | CK- |
|---|---|---|
| Average leaf feeding grade | 1.15 | 7.9 |
| Resistance level | Highly resistant | Susceptible |

(3) Helicoverpa armigera

The transgenic maize event LP026-2 was artificially inoculated at the silking stage of maize for a total of two times, and 20 heads of artificially reared newly hatched larvae were inoculated in maize filament of each maize. 3 days after inoculation, the second inoculation was carried out, with the same number as the first inoculation. 14-21 days after inoculation, the damage rate of female ear, the number of surviving larvae per female ear and the damage length of female ear were investigated one by one. Usually, the investigation begins 14 days after inoculation. If the damage level of negative control material (CK-) reaches susceptible or highly susceptible, it is considered effective, and if it does not reach said level, the investigation could be postponed appropriately, and if after postponement it still does not reach the corresponding level 21 days after inoculation, this inoculation is considered invalid. According to the damage rate of female ear, the number of surviving larvae and the damage length (cm) of female ear, the average damage level of Helicoverpa armigera to female ear in each plot was calculated, and the judgment standard was shown in Table 17. Then, the resistance level of Helicoverpa armigera in maize ear stage was judged according to the standard in Table 18. The resistance results of transgenic maize event LP026-2 to Helicoverpa armigera at the silking stage are shown in FIG. 5 and Table 19. The results showed that transgenic maize event LP026-2 had a high level of resistance to Helicoverpa armigera, and the female ear injury rate, larva survival number, female ear damage length and female ear damage level of transgenic maize event LP026-2 were significantly lower than those of transformation recipient control (CK-).

TABLE 17
Grading criteria of damage degree of female
ear of maize by Helicoverpa armigera

| Damage level of female ear | Description on the symptoms |
|---|---|
| 0 | No damage on female ear |
| 1 | Only the filaments are damaged |
| 2 | Damaged by 1 cm on top of the ear |
| 3+ | For every 1 cm of damage below the |
| | top of the ear, the |
| | corresponding level of damage |
| | increases by 1 level |
| . . . N | |

TABLE 18
Evaluation criteria for resistance of female
ear of maize to Helicoverpa armigera

| Average damage level of female ear | Resistance type |
|---|---|
| 0-1.0 | Highly resistant (HR) |
| 1.1-3.0 | Resistant (R) |
| 3.1-5.0 | Medium resistant (MR) |
| 5.1-7.0 | Susceptible (S) |
| ≥7.1 | Highly susceptible (HS) |

TABLE 19
Resistance results of transgenic maize event LP026-2
to Helicoverpa armigera at silking stage

| Item/plant | LP026-2 | CK- |
|---|---|---|
| Damage rate of female ear (%) | 0 | 100 |
| Larva survival number | 0 | 14 |
| Tunnel length (cm) | 0 | 2.6 |
| Damage level of female ear | 0 | 6.8 |
| Resistance level | Highly resistant | Susceptible |

(4) Conogethes punctiferalis

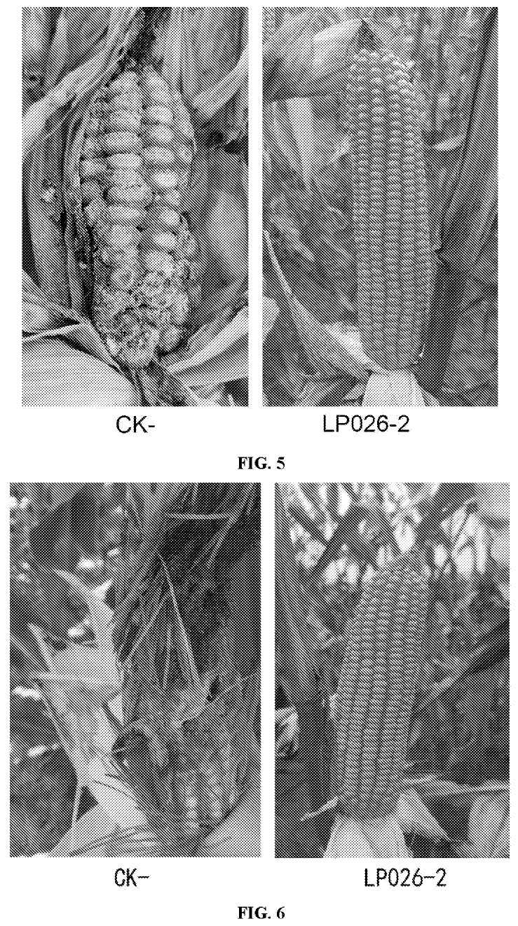

In July 2021, a field natural susceptibility test of Conogethes punctiferalis was carried out in the transgenic maize planting base in Jian'an District, Xuchang City, Henan Province. When 14-21 days after the first pest occurred and the control (CK-) plants were mostly damaged by 4-5 instar higher larvae, the damage rate of Conogethes punctiferalis to maize plants was investigated one by one. The resistance results of transgenic maize event LP026-2 to Conogethes punctiferalis are shown in FIG. 6 and Table 20. The results showed that the damage rate of the Conogethes punctiferalis to the transgenic maize event LP026-2 was significantly lower than that of the control (CK-), which indicated that the transgenic maize event LP026-2 had high resistance to the Conogethes punctiferalis.

TABLE 20
Resistance results of transgenic maize event LP026-2 to
conogethes punctiferalis under natural insect susceptibility conditions

| Item/plant | LP026-2 | CK- |
|---|---|---|
| damage rate(%) | 0 | 73 |

(5) Spodoptera exigua

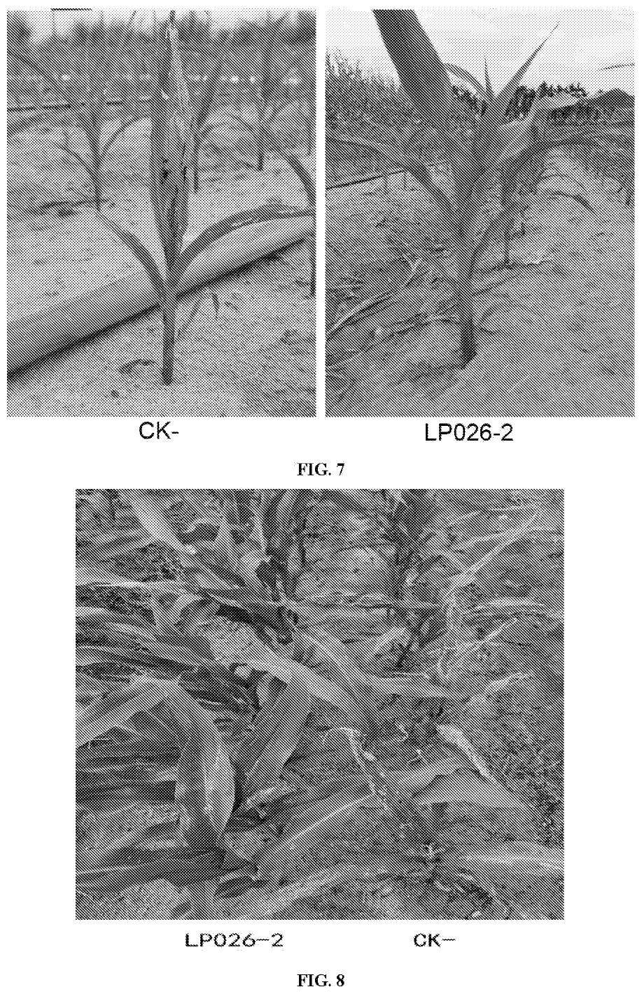

In March 2021, a field natural susceptibility test of Spodoptera exigua was conducted in the transgenic maize planting base in Yazhou District, Sanya City, Hainan Province. The damage rate of Spodoptera exigua to maize plants was investigated one by one when 10-15 days after the first pest occurred and the CK-plants were mostly damaged by 4-6 instar high larvae. The resistance results of transgenic maize event LP026-2 to Spodoptera exigua are shown in FIG. 7 and Table 21. The results showed that under the natural occurrence conditions of Spodoptera exigua, compared with CK-, the damage rate of Spodoptera exigua to transgenic maize event LP026-2 was significantly reduced, which indicated that transgenic maize event LP026-2 had high resistance to Spodoptera exigua.

TABLE 21
Resistance results of transgenic maize event LP026-2 to Spodoptera exigua
under natural insect-susceptible conditions

| Item/plant | LP026-2 | CK- |
|---|---|---|
| damage rate(%) | 0 | 94 |

(6) Spodoptera frugiperda

In March 2021, a field natural susceptibility test of Spodoptera frugiperda was carried out in the transgenic maize planting base in Yazhou District, Sanya City, Hainan Province. When after 10-15 days of the first pest occurred and the CK-plants were mostly damaged by 4-6 instars high larvae, the damage rate of Spodoptera frugiperda on maize plants was investigated one by one. The resistance results of transgenic maize event LP026-2 to Spodoptera frugiperda are shown in Table 22, and the field resistance effect is shown in FIG. 8. The results showed that under the natural occurrence condition of Spodoptera frugiperda, compared with CK-, the damage rate of Spodoptera frugiperda to transgenic maize event LP026-2 was significantly reduced, which indicated that transgenic maize event LP026-2 had high resistance to Spodoptera frugiperda.

TABLE 22
Resistance results of transgenic maize event LP026-2 to
Spodoptera frugiperda under natural insect-susceptible conditions

| Item/plant | LP026-2 | CK- |
|---|---|---|
| damage rate (%) | 0 | 100 |

### Example 6: Herbicide Tolerance Assay for Maize Transformation Events

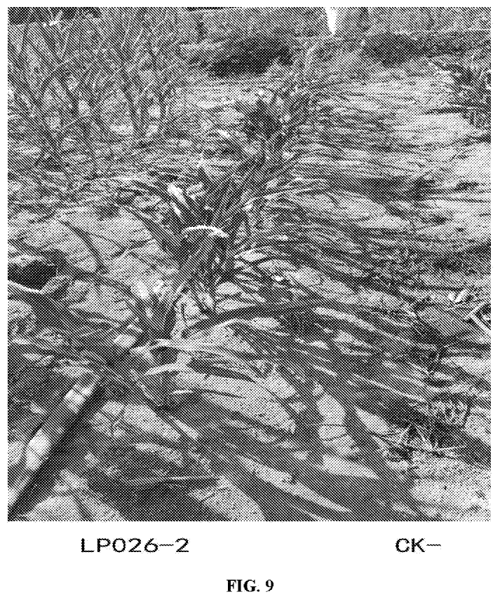

In this experiment, Roundup herbicide (41% glyphosate isopropylammonium salt, aqueous) was selected for spraying. A randomized block design with 3 replicates was used. The plot area was 15m2 (5m×3m), with a row spacing of 60 cm and a plant spacing of 25 cm, under conventional cultivation management, with a 1 m wide isolation zone between plots. The transgenic maize event LP026-2 was subjected to the following 2 treatments: 1) no spraying; 2) spraying Roundup herbicide at the dosage of 1680 g a.e./ha in V3 leaf stage, and then Roundup herbicide was sprayed again at the same dosage in V8 stage. It should be noted that the conversion of glyphosate herbicides with different contents and dosage forms into equivalent glyphosate acid is applicable to the following conclusions. The symptoms of phytotoxicity were investigated 1 week and 2 weeks after application, and the yield of the plot was measured at harvest. The classification of phytotoxicity symptoms is shown in Table 23. The herbicide victimization rate was used as an evaluation index to evaluate the herbicide tolerance in the transformation event, specifically, the herbicide victimization rate (%)=Σ(number of injured plants at the same level×number of levels)/(total number of plants×highest level); wherein the herbicide victimization rate refers to the glyphosate victimization rate, which is determined according to the investigation results of damage by herbicide two weeks after glyphosate treatment. The maize yield of each plot was the total yield (by weight) of maize grains weighed in the middle three rows of each plot, and the yield difference between different treatments was measured in the form of yield percentage, and the yield percentage (%)=sprayed yield/non-sprayed yield. The results of herbicide tolerance and maize yield of transgenic maize event LP026-2 are shown in FIG. 9 and Table 24.

TABLE 23
Grading criteria for the degree of glyphosate herbicide phytotoxicity to maize

| Classification of | |
|---|---|
| phytotoxicity | Description on the symptoms |
| 0 | No phytotoxicity, consistent growth with fresh water control; |
| 1 | Slightly visible symptoms of phytotoxicity, localized color change, with |
| | phytotoxicity spots occupying less than 10% of the leaf area; |
| 2 | Mild growth inhibition or chlorosis, with damage spots accounting for less than ¼ |
| | of the leaf area; |
| 3 | Large impact on growth and development, leaf deformation or plant dwarfing or |
| | damage spots accounting for less than ½ of the leaf area; |
| 4 | Great impact on growth and development, serious deformation of leaves or |
| | obvious dwarfing of plants or less than ¾ of leaf blight spots; |
| 5 | Extremely serious phytotoxicity, plant death or damage spots accounting for more |
| | than ¾ of the leaf area. |

TABLE 24
Tolerance results of transgenic maize event LP026-2
to glyphosate herbicide and yield results of maize

| Item/plant | LP026-2 | CK- |
|---|---|---|
| Glyphosate victimization rate | 0 | 0 |
| (%) (clear water was sprayed) | | |
| Glyphosate victimization rate | 0 | 100 |
| (%) (Roundup 800 ml/mu, recommended use | | |
| concentration 150-250 ml/mu) | | |
| Yield percent % (Roundup 800 ml/mu, | 100 | 0 |
| recommended use concentration | | |
| 150-250 ml/mu) | | |

The results showed that in terms of damage rate by herbicide (glyphosate): 1) the damage rate of transgenic maize event LP026-2 was basically zero under the treatment of glyphosate herbicide (800 ml/mu), and thus, the transgenic maize event LP026-2 had good tolerance to glyphosate herbicide.

And in terms of yield: there was no significant difference in the yield of transgenic maize event LP026-2 under the two treatments of no spraying and spraying 800 ml/mu glyphosate. After spraying glyphosate herbicide, the yield of transgenic maize event LP026-2 basically no decrease, which further indicating that the transgenic maize event LP026-2 possesses a good tolerance to glyphosate herbicide.

In summary, by TaqMan™ analysis, the regenerated transgenic maize plants were tested for the presence of cry1Ab, cry2Ab, cry1Fa and epsps genes and the copy number of insect-resistant and glyphosate herbicide-tolerant lines was characterized (see Example 2). According to the copy number of the target gene, good insect resistance, glyphosate herbicide tolerance and agronomic character performance (see Examples 5 and 6), event LP026-2 was selected as superior by screening, which has single-copy transgene, good insect resistance, glyphosate herbicide tolerance and agronomic character performance (Examples 5 and 6).

Finally, it should be noted that the above Examples are only used to illustrate the technical solutions of the present invention and are not intended to limit it. Although the present invention has been described in detail with reference to the preferred Examples, those skilled in the art should understand that modifications or equivalent substitutions may be made to the technical solutions of the present invention without departing from the spirit and scope of the technical solutions of the present invention.

### INDUSTRIAL APPLICABILITY

The present invention provides a nucleic acid sequence, which comprises one or more selected from the sequences consisting of SEQ ID NOs: 1-7 and the complementary sequences thereof, said nucleic acid sequence is derived from plants, seeds or cells comprising the transgenic maize event LP026-2, and the representative sample of the seeds comprising said event having been deposited under deposit number CCTCC NO: P202207. The transgenic maize event LP026-2 of the present invention not only has good resistance to the feeding by Lepidoptera pests, but is also tolerant to agricultural herbicides containing glyphosate. The maize plant with the dual trait has the following advantages: it is free from economic losses caused by Lepidoptera pests; it is a maize crop tolerate to glyphosate, which is a commonly used commercial herbicide; no reduction in maize yields; and enhanced breeding efficiency with the ability to use molecular markers to track transgene insertion fragments in the breeding population and its progeny. At the same time, the detection method provided by the present invention is capable of identifying the presence of the plant material derived from the transgenic maize event LP026-2 quickly, accurately and consistently, and has good economic value and application prospect.

1. A nucleic acid sequence of transgenic maize event LP026-2, characterized by comprising one or more selected from the sequences consisting of SEQ ID NOs: 1-7 and the complementary sequences thereof.

2. The nucleic acid sequence of claim 1, wherein, the nucleic acid sequence is derived from plants, seeds or cells comprising the transgenic maize event LP026-2, and the representative sample of the seeds comprising the event has been deposited under deposit number CCTCC NO: P202207.

3. The nucleic acid sequence of claim 1, wherein, the nucleic acid sequence is an amplicon for identifying the existence of transgenic maize event LP026-2.

4. A pair of DNA primers comprising a first primer and a second primer, wherein, the first primer and the second primer each comprise a partial sequence of SEQ ID NO:5 or a complementary sequence thereof, and when used in an amplification reaction together with DNA containing a transgenic maize event LP026-2, an amplicon for detecting the transgenic maize event LP026-2 in a sample is created.

5. The pair of primers of claim 4, wherein, the first primer is selected from SEQ ID NO:1 or a complementary sequence thereof, SEQ ID NO:8 or SEQ ID NO: 12; and the second primer is selected from SEQ ID NO:2 or a complementary sequence thereof, SEQ ID NO: 11 or SEQ ID NO:14.

6. The pair of primers of claim 4, wherein, the first primer is selected from SEQ ID NO:1 or a complementary sequence thereof, SEQ ID NO:8 or SEQ ID NO: 12, and the second primer is selected from SEQ ID NO:9 or SEQ ID NO: 13; or the first primer is selected from SEQ ID NO:2 or a complementary sequence thereof, SEQ ID NO:10 or SEQ ID NO:15, and the second primer is selected from SEQ ID NO:11 or SEQ ID NO:14.

7. A DNA probe, characterized by comprising a partial sequence of SEQ ID NO: 5 or a complementary sequence thereof, the DNA probe hybridizes with a DNA molecule containing a nucleic acid sequence selected from the sequences consisting of SEQ ID NOs: 1-7 and the complementary sequences thereof under strict hybridization conditions and does not hybridize with a DNA molecule that does not contain a nucleic acid sequence selected from the sequences consisting of SEQ ID NOs: 1-7 and the complementary sequences thereof under strict hybridization conditions.

8. The probe of claim 7, wherein, the DNA probe comprises a sequence selected from SEQ ID NO:3 or a complementary sequence thereof and SEQ ID NO: 4 or a complementary sequence thereof.

9. The probe of claim 7, wherein, the DNA probe comprises a sequence selected from the group consisting of SEQ ID NO:1 or a complementary sequence thereof, SEQ ID NO:2 or a complementary sequence thereof, SEQ ID NO: 6 or a complementary sequence thereof and SEQ ID NO:7 or a complementary sequence thereof.

10. A marker nucleic acid molecule, characterized by comprising a partial sequence of SEQ ID NO:5 or a complementary sequence thereof, the marker nucleic acid molecule hybridizes with a DNA molecule containing a nucleic acid sequence selected from the sequences consisting of SEQ ID NOs: 1-7 and the complementary sequences thereof under strict hybridization conditions and does not hybridize with a DNA molecule that does not contain a nucleic acid sequence selected from the sequences consisting of SEQ ID NOs: 1-7 and the complementary sequences thereof under strict hybridization conditions.

11. The marker nucleic acid molecule of claim 10, wherein, the marker nucleic acid molecule comprises a sequence selected from SEQ ID NO:3 or a complementary sequence thereof and SEQ ID NO:4 or a complementary sequence thereof.

12. The marker nucleic acid molecule of claim 10, wherein, the marker nucleic acid molecule comprises a sequence selected from the group consisting of SEQ ID NO:1 or a complementary sequence thereof, SEQ ID NO:2 or a complementary sequence thereof, SEQ ID NO:6 or a complementary sequence thereof and SEQ ID NO:7 or a complementary sequence thereof.

13. A method for detecting a DNA containing transgenic maize event LP026-2 in a sample, characterized by comprising:
(1) contacting a sample to be detected with the pair of DNA primers according to claim 4 in a nucleic acid amplification reaction;
(2) carrying out the nucleic acid amplification reaction; and
(3) detecting the amplified product;
wherein the inclusion of a nucleic acid sequence selected from the group consisting of SEQ ID NOs: 1-7 and the complementary sequences thereof in the amplified product indicates the existence of DNA containing transgenic maize event LP026-2 in the detection sample.

14. A method for detecting a DNA containing transgenic maize event LP026-2 in a sample, characterized by comprising:
(1) contacting a sample to be detected with the DNA probe according to claim 7 and/or a marker nucleic acid molecule, wherein the marker nucleic acid molecule comprises a partial sequence of SEQ ID NO:5 or a complementary sequence thereof, the marker nucleic acid molecule hybridizes with a DNA molecule containing a nucleic acid sequence selected from the sequences consisting of SEQ ID NOs: 1-7 and the complementary sequences thereof under strict hybridization conditions and does not hybridize with a DNA molecule that does not contain a nucleic acid sequence selected from the sequences consisting of SEQ ID NOs: 1-7 and the complementary sequences thereof under strict hybridization conditions;
(2) hybridizing the sample to be detected with the probe and/or the marker nucleic acid molecule under strict hybridizing conditions; and
(3) detecting the hybridization of the sample to be detected to the probe and/or the marker nucleic acid molecule.

15. A DNA detection kit, characterized by comprising the pair of DNA primers according to claim 4, a DNA probe, and/or a marker nucleic acid molecule;
wherein the DNA probe comprises a partial sequence of SEQ ID NO:5 or a complementary sequence thereof, the DNA probe hybridizes with a DNA molecule containing a nucleic acid sequence selected from the sequences consisting of SEQ ID NOs: 1-7 and the complementary sequences thereof under strict hybridization conditions and does not hybridize with a DNA molecule that does not contain a nucleic acid sequence selected from the sequences consisting of SEQ ID NOs: 1-7 and the complementary sequences thereof under strict hybridization conditions; and
wherein the marker nucleic acid molecule comprises a partial sequence of SEQ ID NO:5 or a complementary sequence thereof, the marker nucleic acid molecule hybridizes with a DNA molecule containing a nucleic acid sequence selected from the sequences consisting of SEQ ID NOs: 1-7 and the complementary sequences thereof under strict hybridization conditions and does not hybridize with a DNA molecule that does not contain a nucleic acid sequence selected from the sequences consisting of SEQ ID NOs: 1-7 and the complementary sequences thereof under strict hybridization conditions.

16. A method for protecting maize plant from insects, wherein, the transgenic maize plant comprises, sequentially, SEQ ID NO:1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome; a target insect that has ingested transgenic maize plant cells is inhibited from further ingesting the maize plant.

17. A method for protecting maize plant from damage caused by an herbicide, wherein, growing at least one transgenic maize plant comprising a nucleic acid sequence of the transgenic maize event LP026-2, wherein the transgenic maize plant comprises, sequentially, SEQ ID NO:1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome.

18. A method for controlling weeds in a field where maize plants are grown, characterized by comprising applying an effective dose of glyphosate herbicide to the field where at least one transgenic maize plant is grown, wherein the transgenic maize plant comprises, sequentially, SEQ ID NO:1, the nucleic acid sequence at positions 1007-17140 of SEQ ID NO:5 and SEQ ID NO: 2 in its genome; or alternatively, the transgenic maize plant comprises the sequence shown in SEQ ID NO:5 in its genome.

19. A method for cultivating maize plants with insect resistance and/or tolerance to glyphosate herbicide, characterized by comprising: growing at least one maize seed comprising a nucleic acid sequence of transgenic maize event LP026-2;
growing and developing the maize seed into a maize plant;
infecting the maize plant with a target insect and/or spraying the maize plant with an effective dose of glyphosate herbicide, and harvesting the plants with reduced plant damage as compared to other plants without the transgenic maize event LP026-2.

20. A processed product produced from transgenic maize event LP026-2, wherein, the processed product is maize powder, maize flour, maize oil, maize ear silk or maize starch.


SEQUENCE LISTING (39 sequences)

SEQ ID NO: 1 - DNA, 22 bp, synthetic construct
  source 1..22: mol_type = other DNA; note = RB-terminal junction-spanning sequence
catcgaatgcctcaaacactga

SEQ ID NO: 2 - DNA, 22 bp, synthetic construct
  source 1..22: mol_type = other DNA; note = LB-terminal junction-spanning sequence
tttacaccacaagcaattctta

SEQ ID NO: 3 - DNA, 1495 bp, synthetic construct
  source 1..1495: mol_type = other DNA; note = RB-terminal for T-DNA
gcagctatcttcggatggacggatgtaatcaatcaaaaagtcttaggttgagatctttaaggatgaggccattagcccttttcatctgatcagttctttgagagtgtgcaaccgaggcaaaatgcataataattccacattcggtgcaaaaattcatgaactttttagaaagtgaactggcttccattatcagttatggtggttctcggcacaccgaactgattgaagatctctttcgtgaattccactactttggttgataagatcttggctacaagcttagcctcgacccactttgtcaacttatcaacgatcacaaagagatacgtgacttcgcccttcaccggtttgaatgatccaataatgtctaggcaccaagtggagaattgataggtgatagggatagtctaaagttgtgtagctagcatatgagcctggtgtgaaaaacattagcaccccttgcatgtgcgtacgagttgtttggcgtcagttactattgttgatcagaaaaacctgctgataagctttcgccactaacgttttggtgtaagccttagctttttattgaatctgagtcacctaggacttatttgtggggaggactctatccattagatatttgatgaagggagcccaccaaactgactttatagacatcacctcggctagcagatcatcatgagattctagatcttcaaatctggataacttggctggagtaactctgtattattctaaatagttggggcatgcaagatttccacgaagacctctaggggtacaagggaccttgagcagcctaacttttccgggatttcaacttcttcattctttgctcatagaacatatgagaattcaatcccatcgaatttatctttgaggttctgaatctcctggcaataattcatcattcaatccacattgaaagttcaatctttgttgacttggttaattactagcaacgagtcacctagaactcggacccatcgaatgcctcaaacactgatagtttaaactgaaggcgggaaacgacaatctgatcaagagcggagaattaagggagtcacgttatgacccccgccgatgacgcgggacaagccgttttacgtttggaactgacagaaccgcaacgctgcaggaattggccgcaggtggatttgtattaaactaatgactaattagtggcactagcctcaccgacttcgcagacgaggccgctaagtcgcagctacgctctcaacggcactgactaggtagtttaaacgtgcacttaattaaggtaccgggaatttaaatcccgggaggtctcgcagacctagctagttagaatcccgagacctaagtgactagggtcacgtgaccctagtcacttaaagctgatctagtaacatagatgacaccgcgcgcgataatttatcctagtttgcgcgctatattttgttttctatcgcgtattaaatgtataattgcgggactctaatcataaaaacccatc

SEQ ID NO: 4 - DNA, 590 bp, synthetic construct
  source 1..590: mol_type = other DNA; note = LB-terminal for T-DNA
gcgcgcaaactaggataaattatcgcgcgcggtgtcatctatgttactagatccctgcagggaattcttaattaagtgcacgcggccgcctacttagtcaagagcctcgcacgcgactgtcacgcggccaggatcgcctcgtgagcctcgcaatctgtacctagtttagctagttaggacgttaacagggacgcgcctggccgtatccgcaatgtgttattaagttgtctaagcgtcaatttgtttacaccacaagcaattcttaagctgagcaacatggcctcatactgagcaatattattggttgtaggaaagtgtatttgtagtatgtacctgaatttctcaccttttggtgaagtaacaacaactcacgctcttgctccactaagctataaaggtccatcaaatttgactgtccagtgatcaaggggcttcatttcgattaggacgtgtatctcggtcaactttgcaacaaaatcactaagagtcttggccttgatcaccatacgaggtttgaatataatgtcatatactcctagctcaatagcacacttggcaactcaggctgtaacatatttcttgtgcagtac

SEQ ID NO: 5 - DNA, 17487 bp, synthetic construct
  source 1..17487: mol_type = other DNA; note = T-DNA full-length sequence
gcagctatcttcggatggacggatgtaatcaatcaaaaagtcttaggttgagatctttaaggatgaggccattagcccttttcatctgatcagttctttgagagtgtgcaaccgaggcaaaatgcataataattccacattcggtgcaaaaattcatgaactttttagaaagtgaactggcttccattatcagttatggtggttctcggcacaccgaactgattgaagatctctttcgtgaattccactactttggttgataagatcttggctacaagcttagcctcgacccactttgtcaacttatcaacgatcacaaagagatacgtgacttcgcccttcaccggtttgaatgatccaataatgtctaggcaccaagtggagaattgataggtgatagggatagtctaaagttgtgtagctagcatatgagcctggtgtgaaaaacattagcaccccttgcatgtgcgtacgagttgtttggcgtcagttactattgttgatcagaaaaacctgctgataagctttcgccactaacgttttggtgtaagccttagctttttattgaatctgagtcacctaggacttatttgtggggaggactctatccattagatatttgatgaagggagcccaccaaactgactttatagacatcacctcggctagcagatcatcatgagattctagatcttcaaatctggataacttggctggagtaactctgtattattctaaatagttggggcatgcaagatttccacgaagacctctaggggtacaagggaccttgagcagcctaacttttccgggatttcaacttcttcattctttgctcatagaacatatgagaattcaatcccatcgaatttatctttgaggttctgaatctcctggcaataattcatcattcaatccacattgaaagttcaatctttgttgacttggttaattactagcaacgagtcacctagaactcggacccatcgaatgcctcaaacactgatagtttaaactgaaggcgggaaacgacaatctgatcaagagcggagaattaagggagtcacgttatgacccccgccgatgacgcgggacaagccgttttacgtttggaactgacagaaccgcaacgctgcaggaattggccgcaggtggatttgtattaaactaatgactaattagtggcactagcctcaccgacttcgcagacgaggccgctaagtcgcagctacgctctcaacggcactgactaggtagtttaaacgtgcacttaattaaggtaccgggaatttaaatcccgggaggtctcgcagacctagctagttagaatcccgagacctaagtgactagggtcacgtgaccctagtcacttaaagctgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaattcccatggggatcagagctcctatcagtacagcggcgagatgttagttggcaccagcatgatgttcatgaggtcgaactgggtgccagagttcagggtcacgttgatgtccagcgggacgtcggagttgctgctggccaccacgttgccaatgttgatgtcgctgaagcgggcgccgttgtcgttgacgccatcattgttggtcgtcgtgttcacattggtggctgtgtacaccctcccgttgatggtgaccctgatggtggagttgccaatggagctgacgcgcaggtacaggttgtagctgttgccgttgccgcgcagggtgtacctggcggtggtgttgttctgctcgaacctcagggagtcgccctggttgccgaacttctcggagatgaaggtgcgtgtctggttgttcacttgggtggcgtggattggagagatggtgaagccggtgtaatcattgggcgccaggtggatcatggagccgttctcatgcacagcgtggatgttgttcttcctgttatggacgctcaccatgtacgcccttgcacctccgggcgtcccggacggagaggcgatgttcctgatctcgttgtagtgcagtggacggcggaggtcctcgttgcggacgacgagaggaacaccagagatgttcctgatgaagtagtcggggaagtagttagaatttccacgcgccgtgaaggcgccgctccggaggccaagggtggtctcgaaggactcggtttgccagttggtgtcggtggccacgccctcgcggtcggagccgctgtcgagccaggacctcacgaacggggtgagcagcggcggcaggaaggtggagcagttgaagttctggttgaacggcgatgcaccaatgtcgccgctcgagatgccgccggagtagttcactctggcagcaagcagagcatgagttgtggtggagccggggaggccaacaatgttggggaaggtgttggagaggcgagcaccagagaagccgttgaggacgtagttggagttgacttggaacaacgaatacaggaatggccagtcctggctggtgaagctctgagtttgttggggaccagagccgctggcgtagaggttggcgccgctggacaccagcaggctctggtacttgaagagcgaccagatgctgacgtactcgaacacgttcaggaacatgtaggtcctgaactccagcatgtcgtgaagcctcgtattgaggcccttgaaggccgactggtaggtgttgatgcaatagttggagtagtccctggtgtagttcttcaggtagtcgcggtaggtcctcagcgtggctgcagagatgccccactcgtcagcgttgaggatcacgtcacgaatgaaggagaggtgcaggttggcagcctgagcaaagagtggcagcaggagcagctggtagccttgcatctggaactgaggcaagcggttgaggaacagttgttgcatggtgttcacggaagaagtgatggacagaggcaccgcattgcggttggggttgaggaagttgtccacttggcggttgaactcctccacgtttgcttgcagacccgtcagctcagcgttgacgcgagcaagggtatcagtgttgaggcgctggttgagaaacttctcggtctccctgaggatgtcttgcatgaggttggtggagccagatggaaagatcaggttgcggagttccgagaggatgcgcttcccgacgagagagccgaccttcttgagaaggaagctggccaccgtgccgacgacggggtccacgtacaggctgtggttgttcttcttccactccgtccactccttctgaacagtgtcgaggctcttgtgctggaagctgaatggatcatgcgccgcgacgttgtaggcgtcgcagatggtggtgcgaccagagttcaggacggagttgtccatggcctgcatgcagcggatgcgcccgccggtcgacagcggcggcaggtacgacagcgtctcgaacttcttgttgccgtaggccggccacacctgcacgtagtacgtgtgttttggttcgtttggggttgggaattgggatgggatgggcaacacacatcagtccatgcatggatcatcagttcccttcctattactaactcgctagctgcagctgctgcaatgcaaagaactactagctaggatgcatttgttacctgcatgcaccggatccttccgccgttgctgacgttgccgaggcttctggaggagcggcgggcgacggggaggctggcggtggacttgagcccctggaacggagcgacggcggtggccgacgaggccatcatcacggtgggcgccatgctgatcctctagaggccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatcctagaaggattggttgagtatctgatgatccttcaaatgggaatgaatgccttcttatatagagggaattcttttgtggtcgtcactgcgttcgtcatacgcattagtgagtgggctgtcaggacagctcttttccacgttattttgttccccacttgtactagaggaatctgctttatctttgcaataaaggcaaagatgcttttggtaggtgcgcctaacaattctgcaccattccttttttgtctggtccccacaagccagctgctcgatgttgacaagattactttcaaagatgcccactaactttaagtcttcggtggatgtctttttctgaaacttactgaccatgatgcatgtgctggaacagtagtttactttgattgaagattcttcattgatctcctgtagcttttggctaatggtttggagactctgtaccctgaccttgttgaggctttggactgagaattcttccttacaaacctttgaggatgggagttccttcttggttttggcgataccaatttgaataaagtgatatggctcgtaccttgttgattgaacccaatctggaatgctgctaaatcctgagaagcttaacagaggtggatggacagacccgttcttacaccggactgggcgcgggataggatattcagattgggatgggattgagcttaaagccggcgctgagaccatgctcaaggtaggcaatgtcctcagcgtcgagcccggcatctatgtcgagggcattggtggagcgcgcttcggggataccgtgcttgtaactgagaccggatatgaggccctcactccgcttgatcttggcaaagatatttgacgcatttattagtatgtgttaattttcatttgcagtgcagtattttctattcgatctttatgtaattcgttacaattaataaatattaaaatcagattattgactgtcatttgtatcaaatcgtgtttaatggatatttttattataatattgatgatatctcaatcaaaacgtagataataataatatttatttaatatttttgcgtcgcacagtgaaaatctatatgagattacaaaataccgacaacattatttaagatacatagacattaaccctgagactgttggacatcaacgggtagattccttcatgcatagcacctcattcttggggacaaaagcacggtttggccgttccattgctgcacgaacgagctttgctatatcctcgggttggatcatctcatcaggtccaatcaaatttgtccaagaactcatgttagtcgcaacgaaaccggggcatatgtctacgtattactcgagtgtggcagtaactggaatcaactcaaacctgtcaatgtacacttcgttgcctgaactgaaggtatcagcacctactgtgaaactgctctggctcattggaaaggtgaacgcggtgttgatagtggcgtaggagaaagattggaatgtaagtggatcaccggtatccattgtcttgttgaactgaccagcaaagatccgttcacctgcaaccgtaacgtagattcttagattggtagtagaggcatagcgtatcctggcacgatacctttggggaagttgcccattgatgttgacaatggtgtacgcgaatggtcctccactagtgcgtcgaagaatgtctcctcccgtgaaccccggccctcttacaactgtagttcctgactgaagtgtgtgtgccttcaccaagggaatctgagtgattctctctggatcaatggtgtttgtgggggtagcgctacgatgcgtccaagagaacattggtgctctccatgagtcggaacctgagatctcacctggccagcgcacaaaggtaacatgattcagcacatgggagtagtcattccaaggtgcgccgctgttgtcttgaggtggtatctcatctagagagtcaatggtcccggagttcctgaatgtgcgggtgtgattcgtaccagtttgttgaaaggccactcccctaagaccgagtacatagtgaggattgccaaagcctcctcggacgaagacaggatctgacaaggtccgatagaaaggacgtggatcttcatctgcaatccagatggcgcccccgggattgaagaccccgtaactaggaaagttgatacgattgccagccgtgttgcgtgagctaactaagtgtcctccccacacagtttgggatctaacagtctctgcagtcacaaacaaagagttcatgaagtccatgagatggggtggtctgactccaaactcagccctgttgaaaccattgggtatgttcgcagaaactggagagtcttcaatgactgaactggtgtagatctcccttgtaagttgggatgacgtttgaatcggataggtacgaacatcgtagttcggaaagagagcaactatgtctaacacagtaagtgtaaggtctctcctgaactgattgaacctggcccattggcgagtgttagtacctctcaggttctccaatccctgattgtaggtatccaaacaatgtttcgtgtatcgatgaatcagattgatgagtctgttgtagtgattgttgacagtagctatgtccagtccccaaccttgcccaaacgacacagcgtcgcgcagtagtgacaagtgcaggttagcagcttgaacatagaccgagagaagagggatctcgaagctggtaagggtgaagttgttgatggctgtgatcaaagcatcatctgtgttagcaaagcgtatacgcacatcttctctcagttgggcattgttaggattggcttcccactctcttagtgcttcaatgtagatctcatagctgtctgctaagccacgaagggtagtgatggcccgattcctttccaaggtctcaatcctttgttcaatcaactgttcaatctggagaagaaagaggctccaatcagatggagtgatgaagccccagatgaggtcgaagaggccaaacgcaactcccacacctggaacaaactcagacaacaggaaacgtgtaagggacagggagatgtctaacggcaatctgccagtcgacctctcttcgttgagaatctctacttcaggattgttgaggcagttaaagggaatgcactgattctgtatgttgttctccatgagctcctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcagcctaggttaagtgactagggtcacgtgactctagtcacttacttcgtggagatataggggaaagagaacgctgatgtgacaagtgagtgagatatagggggagaaatttagggggaacgccgaacacagtctaaagtagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcaggcgcgcctagcccaggtcctcgttcaggtcggtgcagcccacatcgatgtccaaggagaagtggtggctgtggtgggcacacttgccgatcgggctgggggcgctcagcggccagagggaaccagtaccgggcacgttgacggtctcgtgcttggcgttgtagcggatcaggtaaatctcgaggtcttggctgtcttcgatgtagccgcggagctggtagcgagtgtaagccttgagcttggactcatcgatcttctggtacaagtaggtagggtagcactcgtcgaaagtgcccaggagagtcacgtagttctccttgaacacatcgtcgccgccctggatcgtgatgtcggtgctgccgcgccagccgcggtcgagctgcctgttgatgccgcggaaattggggtcctggaggagattcctctcgtcgctgagacgcttggcatgcttcaccttctcggacagctccttcttctcgtcgaggcagaactcatcggagaggcactccacgaggttggagacttggtcgatgtggtagtcagtgacgtcggtcttcaggccgatctgattgctggacgtgaagagctcattgacagccttctgggctctctccaggtcgtactcggcttcgaaggtgacctcggctggcacgaactcaatgcggtcaatgtacacctcattgccggaattgaacacgtgggcgctcagggtgaaaacgctggagccgttggagaagttgaagggggtggtgaaacccacggtgcggaagctgccggattggaggttgctgccgctggacatggtggcggagaagttaccctgattgatcggcctgccgtcgatggaggtgtggaattgcaggttggtggtgctagcgtagcgaatcctgacgcggtacctctgggacaggggagcggtgatgttgacgcggagggtgctgatctggcccggggaggtcctgcgcaggatgtcgccgcccgtgaagcctgggcccttcaccacggaggtgccgctgcccaggttggtggacttggtgagggggatttgggtgatttgggaggacggaatgatattgttgaactccgcgctgcgatgaatccaggagaacataggagctctgatgatgctcacggacgagttgctgaagccggagcggaacatggacacgtggctgagcctgtgggaaaaaccctgcctggggggcacattgttgttctgtggtgggatctcgtccagggaatccaccgtgccgctcttgcggtagacagcggagggcaggttggaggaggtgccgtaggcgaactcagtgccatccaggacggacagctgctggttgttgataccgatgttgaagggcctgcggtacagggtggagctcagggtgcggtagacgccctggcccagctgagcgacgatgcgttgttgtggagcggcgttgcccatcgtgccgtagagaggaaaggtaaactcggggccgctgaagccgaccggggaggccatgatctggtggccggaccagtagtactcgccgcggtgggcatcggtgtagatagtgatgctgttgaggatgtccatcaggtgtgggctcctgatggagccctcgatgccctgggcgctgcccctgaagctaccgtcgaagttctccaggacggggttggtgtagatttcgcgggtcagttgggacacggtgcggatcgggtaggtgcgggagtcgtagttcgggaagagggacacaatgtccaggacggtgagggtcagctcgcgcctgaactggttgtagcgaatccagtctctagaatcagggccccagacgcgctccaggccagtgttgtaccagcggacagcgtggtcggtgtagttgccgatcagcctggtgaggtcgttgtagcggctgttgatggtggcggcgtcgaagccccacctctggccaaacacgctgacgtccctcagcacgctgaggtgcaggttggcggcctggacgtacacggacaggagcgggacttggtagttctggacggcgaagagtgggatggcggtggtcagggcgctgttcatgtcgttgaactggatgcgcatctcctcgcggagagctgggttagtggggtcggcctcccactcgcggaagctctcagcgtagatttggtagaggttgctgaggccctccaggcggctgatggcctggttcctggcgaactcctcgatcctctggttgatgagctgctcgatttgcaccaggaaggcgtcccactgggaggggccaaagatgccccagatgatgtccacgaggcccaggacgaagccagcgcctggcacgaactcgctgagcaggaactgcgtgagggagagggagatgtcgatgggggtgtaaccggtctcgatgcgctcaccgccgagcacctcgacctcagggttgctgaggcagttgtacgggatgcactcgttgatgtttgggttgttgtccatggctagcttctacctacaaaaaagctccgcacgaggctgcatttgtcacaaatcatgaaaagaaaaactaccgatgaacaatgctgagggattcaaattctacccacaaaaagaagaaagaaagatctagcacatctaagcctgacgaagcagcagaaatatataaaaatataaaccatagtgcccttttcccctcttcctgatcttgtttagcacggcggaaattttaaaccccccatcatctcccccaacaacggcggatcgcagatctacatccgagagccccattccccgcgagatccgggccggatccacgccggcgagagccccagccgcgagatcccgcccctcccgcgcaccgatctgggcgcgcacgaagccgcctctcgcccacccaaactaccaaggccaaagatcgagaccgagacggaaaaaaaaacggagaaagaaagaggagaggggcggggtggttaccggcggcggcggaggcctcccttggatcttatggtgtgttgtccctgtgtgttctccaatagtgtggcttgagtgtgtggaagatggttctagaggatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggggcgcgcctactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgaactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcagggaattcttaattaagtgcacgcggccgcctacttagtcaagagcctcgcacgcgactgtcacgcggccaggatcgcctcgtgagcctcgcaatctgtacctagtttagctagttaggacgttaacagggacgcgcctggccgtatccgcaatgtgttattaagttgtctaagcgtcaatttgtttacaccacaagcaattcttaagctgagcaacatggcctcatactgagcaatattattggttgtaggaaagtgtatttgtagtatgtacctgaatttctcaccttttggtgaagtaacaacaactcacgctcttgctccactaagctataaaggtccatcaaatttgactgtccagtgatcaaggggcttcatttcgattaggacgtgtatctcggtcaactttgcaacaaaatcactaagagtcttggccttgatcaccatacgaggtttgaatataatgtcatatactcctagctcaatagcacacttggcaactcaggctgtaacatatttcttgtgcagtac

SEQ ID NO: 6 - DNA, 500 bp, synthetic construct
  source 1..500: mol_type = other DNA; note = LP026 T-DNA sequence
tcaaacactgatagtttaaactgaaggcgggaaacgacaatctgatcaagagcggagaattaagggagtcacgttatgacccccgccgatgacgcgggacaagccgttttacgtttggaactgacagaaccgcaacgctgcaggaattggccgcaggtggatttgtattaaactaatgactaattagtggcactagcctcaccgacttcgcagacgaggccgctaagtcgcagctacgctctcaacggcactgactaggtagtttaaacgtgcacttaattaaggtaccgggaatttaaatcccgggaggtctcgcagacctagctagttagaatcccgagacctaagtgactagggtcacgtgaccctagtcacttaaagctgatctagtaacatagatgacaccgcgcgcgataatttatcctagtttgcgcgctatattttgttttctatcgcgtattaaatgtataattgcgggactctaatcataaaaacccatc

SEQ ID NO: 7 - DNA, 254 bp, synthetic construct
  source 1..254: mol_type = other DNA; note = LP026 T-DNA sequence
gcgcgcaaactaggataaattatcgcgcgcggtgtcatctatgttactagatccctgcagggaattcttaattaagtgcacgcggccgcctacttagtcaagagcctcgcacgcgactgtcacgcggccaggatcgcctcgtgagcctcgcaatctgtacctagtttagctagttaggacgttaacagggacgcgcctggccgtatccgcaatgtgttattaagttgtctaagcgtcaatttgtttacaccaca

SEQ ID NO: 8 - DNA, 23 bp, synthetic construct
  source 1..23: mol_type = other DNA; note = Primer 11, the first primer for amplifying SEQ ID NO:3
gcagctatcttcggatggacgga

SEQ ID NO: 9 - DNA, 28 bp, synthetic construct
  source 1..28: mol_type = other DNA; note = Primer 12, the second primer for amplifying SEQ ID NO:3
gatgggtttttatgattagagtcccgca

SEQ ID NO: 10 - DNA, 29 bp, synthetic construct
  source 1..29: mol_type = other DNA; note = Primer 13, the first primer for amplifying SEQ ID NO:4
gcgcgcaaactaggataaattatcgcgcg

SEQ ID NO: 11 - DNA, 31 bp, synthetic construct
  source 1..31: mol_type = other DNA; note = Primer 14, the second primer for amplifying SEQ ID NO:4
gtactgcacaagaaatatgttacagcctgag

SEQ ID NO: 12 - DNA, 25 bp, synthetic construct
  source 1..25: mol_type = other DNA; note = Primer 15, a primer for the 5' flanking genome
gagatacgtgacttcgcccttcacc

SEQ ID NO: 13 - DNA, 27 bp, synthetic construct
  source 1..27: mol_type = other DNA; note = Primer 16, a primer for T-DNA paired with SEQ ID NO: 12
atcagctttaagtgactagggtcacgt

SEQ ID NO: 14 - DNA, 26 bp, synthetic construct
  source 1..26: mol_type = other DNA; note = Primer 17, a primer for 3' flanking genome
gttgaccgagatacacgtcctaatcg

SEQ ID NO: 15 - DNA, 24 bp, synthetic construct
  source 1..24: mol_type = other DNA; note = Primer 18, a primer for T-DNA paired with SEQ ID NO: 14
ccgcctacttagtcaagagcctcg

SEQ ID NO: 16 - DNA, 21 bp, synthetic construct
  source 1..21: mol_type = other DNA; note = Primer 1 for Taqman detection of Cry1Ab
tgggaggacggaatgatattg

SEQ ID NO: 17 - DNA, 21 bp, synthetic construct
  source 1..21: mol_type = other DNA; note = Primer 2 for Taqman detection of Cry1Ab
aactcgtccgtgagcatcatc

SEQ ID NO: 18 - DNA, 22 bp, synthetic construct
  source 1..22: mol_type = other DNA; note = Probe 1 for Taqman detection of Cry1Ab
aactccgcgctgcgatgaatcc

SEQ ID NO: 19 - DNA, 18 bp, synthetic construct
  source 1..18: mol_type = other DNA; note = Primer 3 for Taqman detection of Cry2Ab
ggacagaggcaccgcatt

SEQ ID NO: 20 - DNA, 18 bp, synthetic construct
  source 1..18: mol_type = other DNA; note = Primer 4 for Taqman detection of Cry2Ab
cgggtctgcaagcaaacg

SEQ ID NO: 21 - DNA, 24 bp, synthetic construct
  source 1..24: mol_type = other DNA; note = Probe 2 for Taqman detection of Cry2Ab
tccacttggcggttgaactcctcc

SEQ ID NO: 22 - DNA, 21 bp, synthetic construct
  source 1..21: mol_type = other DNA; note = Primer 5 for Taqman detection of Cry1Fa
gctatgtccagtccccaacct

SEQ ID NO: 23 - DNA, 22 bp, synthetic construct
  source 1..22: mol_type = other DNA; note = Primer 6 for Taqman detection of Cry1Fa
caagctgctaacctgcacttgt

SEQ ID NO: 24 - DNA, 21 bp, synthetic construct
  source 1..21: mol_type = other DNA; note = Probe 3 for Taqman detection of Cry1Fa
cccaaacgacacagcgtcgcg

SEQ ID NO: 25 - DNA, 20 bp, synthetic construct
  source 1..20: mol_type = other DNA; note = Primer 7 for Taqman detection of EPSPS
gcaaatcctctggcctttcc

SEQ ID NO: 26 - DNA, 19 bp, synthetic construct
  source 1..19: mol_type = other DNA; note = Primer 8 for Taqman detection of EPSPS
tgaaggaccggtgggagat

SEQ ID NO: 27 - DNA, 18 bp, synthetic construct
  source 1..18: mol_type = other DNA; note = Probe 4 for Taqman detection of EPSPS
cgtccgcattcccggcga

SEQ ID NO: 28 - DNA, 23 bp, synthetic construct
  source 1..23: mol_type = other DNA; note = A first primer 9 for endogenous gene zSSIIb of maize
agcagacggcacggcatctctgt

SEQ ID NO: 29 - DNA, 23 bp, synthetic construct
  source 1..23: mol_type = other DNA; note = A second primer 10 for endogenous gene zSSIIb of maize
cagaagtagaactaccgggccct

SEQ ID NO: 30 - DNA, 538 bp, synthetic construct
  source 1..538: mol_type = other DNA; note = Probe for Cry1Ab in Southern hybridization assay
catccagttcaacgacatgaacagcgccctgaccaccgccatcccactcttcgccgtccagaactaccaagtcccgctcctgtccgtgtacgtccaggccgccaacctgcacctcagcgtgctgagggacgtcagcgtgtttggccagaggtggggcttcgacgccgccaccatcaacagccgctacaacgacctcaccaggctgatcggcaactacaccgaccacgctgtccgctggtacaacactggcctggagcgcgtctggggccctgattctagagactggattcgctacaaccagttcaggcgcgagctgaccctcaccgtcctggacattgtgtccctcttcccgaactacgactcccgcacctacccgatccgcaccgtgtcccaactgacccgcgaaatctacaccaaccccgtcctggagaacttcgacggtagcttcaggggcagcgcccagggcatcgagggctccatcaggagcccacacctgatggacatcctcaacagcatcactatctacaccgatgcccac

SEQ ID NO: 31 - DNA, 597 bp, synthetic construct
  source 1..597: mol_type = other DNA; note = Probe for Cry2Ab in Southern hybridization assay
agaagaacaaccacagcctgtacgtggaccccgtcgtcggcacggtggccagcttccttctcaagaaggtcggctctctcgtcgggaagcgcatcctctcggaactccgcaacctgatctttccatctggctccaccaacctcatgcaagacatcctcagggagaccgagaagtttctcaaccagcgcctcaacactgatacccttgctcgcgtcaacgctgagctgacgggtctgcaagcaaacgtggaggagttcaaccgccaagtggacaacttcctcaaccccaaccgcaatgcggtgcctctgtccatcacttcttccgtgaacaccatgcaacaactgttcctcaaccgcttgcctcagttccagatgcaaggctaccagctgctcctgctgccactctttgctcaggctgccaacctgcacctctccttcattcgtgacgtgatcctcaacgctgacgagtggggcatctctgcagccacgctgaggacctaccgcgactacctgaagaactacaccagggactactccaactattgcatcaacacctaccagtcggccttcaagggcctcaatacgaggcttcacgaca

SEQ ID NO: 32 - DNA, 425 bp, synthetic construct
  source 1..425: mol_type = other DNA; note = Probe for Cry1Fa in Southern hybridization assay
acccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctga

SEQ ID NO: 33 - DNA, 655 bp, synthetic construct
  source 1..655: mol_type = other DNA; note = Probe for EPSPS in Southern hybridization assay
ctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgatt

SEQ ID NO: 34 - DNA, 23 bp, synthetic construct
  source 1..23: mol_type = other DNA; note = Primer for T-DNA in the same orientation as SEQ ID NO:13
ctaacatctcgccgctgtactga

SEQ ID NO: 35 - DNA, 20 bp, synthetic construct
  source 1..20: mol_type = other DNA; note = Primer for T-DNA in the opposite orientation as SEQ ID NO:13
cagagttcagggtcacgttg

SEQ ID NO: 36 - DNA, 20 bp, synthetic construct
  source 1..20: mol_type = other DNA; note = Primer for T-DNA in the opposite orientation as SEQ ID NO:13
gccaatgttgatgtcgctga

SEQ ID NO: 37 - DNA, 20 bp, synthetic construct
  source 1..20: mol_type = other DNA; note = Primer for T-DNA in the same orientation as SEQ ID NO:15
cgatacgaaggctgcctgaa

SEQ ID NO: 38 - DNA, 20 bp, synthetic construct
  source 1..20: mol_type = other DNA; note = Primer for T-DNA in the opposite orientation as SEQ ID NO:15
ccatcaggtccatgaactcc

SEQ ID NO: 39 - DNA, 20 bp, synthetic construct
  source 1..20: mol_type = other DNA; note = Primer for T-DNA in the opposite orientation as SEQ ID NO:15
gtgacagggttttccgacac